SEQUENCE LISTING (23 sequences)

SEQ ID NO: 1 - protein, 10 aa, Artificial
  Example 1, Sequence a
KIKVYLPRMK

SEQ ID NO: 2 - protein, 10 aa, Artificial
  Example 1, Sequence b
VFKGLWEKAF

SEQ ID NO: 3 - protein, 10 aa, Artificial
  Example 1, Sequence c
PVQMMYQIGL

SEQ ID NO: 4 - protein, 10 aa, Artificial
  Example 1, Sequence d
AADQARELIN

SEQ ID NO: 5 - protein, 10 aa, Artificial
  Example 1, Sequence e
GSIGAASMEF

SEQ ID NO: 6 - protein, 13 aa, Artificial
  Example 1, Sequence f
VFKGLWEKAFKDE

SEQ ID NO: 7 - protein, 13 aa, Artificial
  Example 1, Sequence g
KIKVYLPRMKMEE

SEQ ID NO: 8 - protein, 13 aa, Artificial
  Example 1, Sequence h
FSLASRLYAEERY

SEQ ID NO: 9 - protein, 13 aa, Artificial
  Example 1, Sequence i
VDAASVSEEFRAD

SEQ ID NO: 10 - protein, 13 aa, Artificial
  Example 1, Sequence j
RIMGEQEQYDSYN

SEQ ID NO: 11 - protein, 16 aa, Artificial
  Example 1, Sequence k
ERKIKVYLPRMKMEEK

SEQ ID NO: 12 - protein, 16 aa, Artificial
  Example 1, Sequence l
VQHFKRELMNLPQQCN

SEQ ID NO: 13 - protein, 16 aa, Artificial
  Example 1, Sequence m
GLFRVASMASEKMKIL

SEQ ID NO: 14 - protein, 16 aa, Artificial
  Example 1, Sequence n
AEAGVDAASVSEEFRA

SEQ ID NO: 15 - protein, 16 aa, Artificial
  Example 1, Sequence o
MLVLLPDEVSGLEQLE

SEQ ID NO: 16 - protein, 6 aa, Artificial
  Example 1, Sequence of peptide number 42
GPARLA

SEQ ID NO: 17 - protein, 6 aa, Artificial
  Example 1, Sequence of peptide 43
GVPFGP

SEQ ID NO: 18 - protein, 15 aa, Artificial
  Example 1, Sequence if peptide 44
GGSGPFGRSALVPEE

SEQ ID NO: 19 - protein, 15 aa, Artificial
  Example 1, Sequence of peptide 45
GGSGPDGRSALVPEE

SEQ ID NO: 20 - protein, 15 aa, Artificial
  Example 1, Sequence of peptide 46
GGSGPFGRSDLVPEE

SEQ ID NO: 21 - protein, 7 aa, Artificial
  Example 1, Sequence of peptide 47
PAPFAAA

SEQ ID NO: 22 - protein, 8 aa, Artificial
  Example 1, Sequence of peptide 48
GPARLAIG

SEQ ID NO: 23 - protein, 384 aa, Homo sapiens
GSIGAASMEFCFDVFKELKVHHANENIFYCPIAIMSALAMVYLGAKDSTRTQINKVVRFDKLPGFGDSIEAQCGTSVNVHSSLRDILNQTKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

(19) United States
(12) Patent Application Publication
(10) Pub. No.: US 2009/0258381 A1
(43) Pub. Date: Oct. 15, 2009

(54) Methods for Determining the Cleavability of Substrates

(76) Inventors: Hans-Heiner Gorris, Bedburg-Hau (DE); Steffen Bade, Klein Rönnau (DE); Niels Röckendorf, Bargfeld-Stegen (DE); Andreas Frey, Gross Niendorf (DE)

Correspondence Address: NIXON & VANDERHYE, PC, 901 NORTH GLEBE ROAD, 11TH FLOOR, ARLINGTON, VA, 22203, US

(21) Appl. No.: 12/084,204

(22) PCT Filed: Oct. 31, 2006
(86) PCT No.: PCT/EP2006/010490

§ 371 Date: Apr. 28, 2008

(30) Foreign Application Priority Data
Oct. 31, 2005 (DE) 102005051978.4

(51) Int. Cl. C12Q 1/37; G01N 33/531; C07K 17/00

(52) U.S. Cl. 435/23; 435/4; 530/300

(57) ABSTRACT

The invention relates to methods for examining the enzymatic cleavability of substrates. In the methods, compounds, which have a cleavability of the section to be examined, are firstly synthesized on a first solid phase, separated therefrom, the cleavage reaction is carried out in solution and the cleaved and uncleaved compounds are immobilized on a second solid phase and the cleavability is determined.

The present invention relates to methods and the compounds necessary therefor for investigation of the enzymatic cleavability of substrates.

Enzyme-catalyzed degradation processes play a central role in all organisms. Enzymes enable and expedite a large number of biological reactions and metabolic processes. Those substances whose conversion is catalyzed by the enzymes are generally referred to as substrates. Enzymes bind their substrates highly specifically and exclusively to their catalytic center, either by the “lock and key principle” or by the “induced fit principle”. This means that a particular enzyme only accepts as substrate a very restricted number of substances which comply with the steric requirements of binding in the catalytic center (substrate specificity). Each enzyme catalyzes only very particular, defined reactions (action specificity), and the rate of conversion depends on the so-called catalytic efficiency (k_{cat}/K_{M}). The action of enzymes can be reduced or abolished by so-called inhibitors.

Proteases or peptidases are enzymes which catalyze the hydrolytic degradation of proteins or peptides. They are assigned to various classes depending on their catalytic mechanism, and of these the serine proteases have been best investigated. For example, they include the small-bowel digestive enzyme trypsin, chymotrypsin and elastase. A feature distinguishing between the proteases relates to the cleavage site of the substrate. Exopeptidases cleave proteins or protein fragments at the free ends by continuously liberating amino acids from the ends. With endopeptidases, in contrast, the cleavage takes place within the peptide chain. New cleavage possibilities for exopeptidases are created in this way. Because of the central role played by proteins in a living creature, proteases also have great importance. Under- or overproduction of proteases, and disturbances in the control of their enzymatic action may cause serious disorders. The deficiency associated with underproduction is compensated by protease supplementation, while protease inhibitors are employed for hyperfunction of enzymes. Use of protease inhibitors is also appropriate for the therapy of infectious diseases in which proteases of the pathogen are necessary for the infection and its propagation in the host. However, if the pathogens such as, for example, the HI virus (AIDS) or Plasmodium falciparum (malaria) show a strong genetic drift, resistance is rapidly developed against particular inhibitors, and new active substances must be developed.

The activity of proteases must moreover be taken into account when developing medicaments based on peptides or proteins. This especially applies when they are intended for oral administration. It is necessary in this case for active substances to be designed so that they are not attacked by proteases of the digestive tract. Suitable test systems are necessary both for developing peptide active substances for oral administration and for developing protease inhibitors, to make it possible to test the stability of peptides toward proteases in the absence or presence of protease inhibitors.

Various approaches to the investigation of the cleavability of substrates and of enzyme specificities are known.

Although computer simulations and theoretical models for the binding of a substrate in the catalytic center of the protease and for the mechanistic progress of protease-catalyzed reactions have existed for a long time, there is a lack of suitable methods for practical investigation of the protease-substrate interaction. Various approaches have been proposed.

Larger substrates and whole proteins have been brought into contact with a protease mixture, and the cleavage fragments have then been separated by electrophoresis or chromatography. The disadvantage of these methods is, however, that accurate knowledge about the fragments to be expected is necessary if the detection is to take place with the aid of antibodies. Accurate separation and identification of the fragments has been possible with reverse phase high performance liquid chromatography (RP-HPLC) in combination with mass spectrometry methods (Coombs et al., 1996). However, a mass spectrometer must be available for measuring mass spectra for every single determination of enzyme kinetics, which is not the case with simply equipped laboratories.

Immunological methods have been used in various formats for measurements of enzyme activity. Haber et al. (1969) developed a competitive radioimmunoassay (RIA), which is still used in modified form in clinical diagnosis, for determining the blood plasma protease renin, a marker of certain types of hypertension. In this case, a defined amount of the renin substrate angiotensinogen is added to the blood plasma sample, and the angiotensin formed is determined as competitor of radiolabeled angiotensin in the radioimmunoassay. The absolute plasma renin concentration can be ascertained by comparison with a renin calibration solution.

In more recent methods, the radiolabeling has generally been replaced by an enzymatic amplification system which converts a color substrate which is measured by photometry.

To determine HIV-1 protease activity, biotin-labeled peptide substrates have been bound via streptavidin to a microtiter plate. After completion of the cleavage by the HIV-1 protease, only the uncut, but not the cleaved, peptides were detected by an antibody and an enzyme-coupled second antibody (O. Gutiérrez et al., 2002).

Such methods, in which an enzyme acts in solution on a solid phase-bound substrate, and which are disclosed for example in German patent application DE 101 187 74, are referred to as heterogeneous enzyme assays (HEA). These simple and robust methods, with which it is also possible to assay unpurified enzyme fractions, have the disadvantage, however, that free accessibility of the enzyme to the solid phase-bound substrate is not ensured. Only limited conclusions about enzyme kinetics are possible. In addition, only specific enzymatic activities can be investigated, where the cleavage site is accurately defined. In the case of enzymes with a broad substrate range, such as, for example, the small-bowel digestive enzymes, it is not possible to preclude degradation of the protein for binding the substrate to the solid phase by the enzymatic activity. As a consequence, the uncleaved peptides would no longer be detectable either. Finally, it is necessary for each substrate to be investigated either to prepare a new detection antibody, or the recognition sequence for the antibody must be attached to the respective substrate.

Methods for investigating enzyme kinetics require accurate estimation of the substrate conversion. A critical point for investigating enzymatic processes is the availability of suitable substrates. Only if the decrease in the substrate concentration or the increase in the product concentration can be followed directly, conclusions about enzyme kinetics are possible. In the case of the hydrolysis of proteins and peptides, there are experimental difficulties in distinguishing substrate and product and in detecting changes in concentration between these two molecules.

In indirect methods such as, for example, biochemical or immunochemical fragment analysis, the homogeneous immunoprotease assay, the heterogeneous immunoprotease assay, and the protease assay by enzyme fragment complementation, the position of the cleavage and the extent of the interaction between protease and substrate can be ascertained only by subsequent procedures.

In biochemical or immunochemical fragment analysis, the cleavage products of a proteolysis reaction are isolated by chromatography or separated by electrophoresis and then characterized individually, e.g. by sequencing, mass spectrometry or immunoblotting (e.g. Coombs et al., 1996). The method is employed in protein sequencing and is appropriate if the effect of a purified protease on a pure substrate is to be investigated or if antibodies against a putative protease cleavage site of the substrate exist. Cleavage kinetics can only be carried out in special cases and with great effort, because a product purification or separation must be carried out for each measurement point, and the resulting fragments are often of similar size and are not amenable to complete separation or purification. In addition, complex intermediate mixtures may result if the substrate has a plurality of cleavage sites for the protease. In such cases, immunological detection is also very complicated because a separate antibody must be available for each cleavage site. These separation problems are multiplied if the effect of protease mixtures or complex protease-containing biological samples on a substrate is to be determined, because the proteases in this case also attack themselves or likewise cleave irrelevant substrates present in the sample. In this case, the product may in some circumstances be concealed by thousands of irrelevant fragments of similar size.

In the homogeneous immunoprotease assay, the proteolytic decomposition or formation of a defined substrate or product is determined with the aid of an immunoassay by initially exposing the substrate to the protease in solution, and then determining the product or substrate present in the reaction mixture either directly or as competitor in an immunoassay (e.g. RIA or ELISA). The substrate or product need not be purified for this purpose, and crude enzyme preparations can be employed. However, a product- or substrate-specific antibody must always exist in order to carry out the RIA or ELISA which follows the proteolysis. This means that an immunoprotease assay is always designed specifically for a substrate-enzyme pair. Haber et al. (1969) developed, for example, a competitive radioimmunoassay (RIA), which is still used in modified form in clinical diagnosis, for determining the blood plasma protease renin, a marker of certain types of hypertension. In this case, a defined amount of the renin substrate angiotensinogen is added to the blood plasma sample, and the angiotensin formed is determined as competitor of radiolabeled angiotensin in the radioimmunoassay. The absolute plasma renin concentration can be ascertained by comparison with a renin calibration solution.

In the heterogeneous immunoprotease assay, the substrate is already in immobilized form, and the substrate remaining after the reaction is ascertained by a substrate-specific antibody. This simple and robust method with which it is also possible to assay unpurified enzyme fractions, has the disadvantage, however, that free accessibility of the enzyme to the solid phase-bound substrate is not ensured. Only limited conclusions about enzyme kinetics are possible. In addition, only specific enzymatic activities where the cleavage site is accurately defined can be investigated. In the case of enzymes with a broad substrate range, such as, for example, the small-bowel digestive enzymes, it is not possible to preclude degradation of the anchoring of the substrate to the solid phase by the enzymatic activity as well. As a consequence, the uncleaved peptides would no longer be detectable either. Finally, it is necessary for each substrate to be investigated either to prepare a new detection antibody, or the recognition sequence for the antibody must be attached to the respective substrate. This means that this assay format is, like the homologous immunoprotease assay, designed for one substrate-enzyme pair.

One example of this assay format was disclosed in German patent application DE 101 187 74. Another one was described by Gutiérrez et al. (2002) for analyzing HIV-1 protease activity. In this case, biotin-labeled peptide substrates were bound via streptavidin to a microtiter plate. After completion of the cleavage by the HIV-1 protease, only the uncut, but not the cleaved, peptides were detected by an antibody and an enzyme-coupled second antibody.

Direct methods such as, for example, the chromogenic enzyme assay or enzyme assays based on intramolecular fluorescence quenching allow the protease reaction to be analyzed in real time and are therefore well suited for kinetic investigations.

The chromogenic enzyme assay does not, however, provide any information about the cleavability of amino acid sequence motifs, but is used to determine enzyme activities and quantities, because a chromogenic group, not an amino acid, is always present in the P1′ position of a putative cleavage site. Methods and compounds for carrying out such chromogenic enzyme assays have long been known to the skilled worker (e.g. Bender et al., 1966).

In enzyme assays based on intramolecular fluorescence quenching, this disadvantage of chromogenic enzyme assays is avoided, because the labels necessary for detection are located on the proximal and distal end of a linear synthetic peptide substrate. The protease recognition sequence encompassed by the labels can be chosen without restriction and may thus represent a complete naturally occurring substrate sequence. This assay format is based on the so-called FRET technology (fluorescence resonance electron transfer). FRET is a distance-dependent interaction between the electron-excited states of two dye molecules, in which energy is transferred from a donor molecule to an acceptor molecule without light being emitted. The presence of the acceptor quenches the emission of the donor. The first applications of FRET-based enzyme substrates go back to the 1970s (Latt et al., 1972, and Yaron et al., 1979). The dye molecules are attached at both ends of the linear peptide chain. As long as the peptide is uncleaved, the donor produces no fluorescent signal. On cleavage of the peptide, donor and acceptor are spatially separated from one another, the quenching is abolished, and the donor emits a light signal which is determined by photometry. A substantially free choice of the cleavage site with the flanking sequences is possible with this method as long as the dye molecules enclose the cleavage site.

However, even the FRET method has various disadvantages. Thus, the efficiency of the radiationless energy transfers greatly depends on the distance between donor and acceptor. It decreases with the sixth power of the intramolecular distance, so that to date only peptide substrates having a maximum of 11 amino acids have been used (Wang et al., 1993). A further problem is the poor water solubility of most fluorescent dyes and of acceptors, so that—depending on the peptide sequence—an organic solvent such as dimethyl sulfoxide must be added to the assay buffer. This may have an influence on the enzyme activity.

It is furthermore possible under certain conditions that there is interference with or superimposition on the excitation of the donor or the emission of the acceptor. Crude protease preparations often contain biogenic dyes or fluorophores, and even the substrate sequences themselves may comprise fluorescent amino acids such as, for example, tryptophan. The latter in particular is a problem for high-throughput investigations of peptide substrate libraries because exclusion of certain candidates a priori introduces an unwanted weighting into the method. In order to avoid this, it would be necessary to investigate such substrate molecules separately, involving considerable additional effort (J. George et al. 2003).

Combinatorial chemistry makes it possible to synthesize a large number of different peptide substrates in parallel. A customary method for preparing such libraries is the spot synthesis technique (Frank, 1992). The peptide substrates are bound to a surface on one side, and on the other side they can be provided with a label by which it is possible to examine the cleavage. The label used is a radioactive isotope, a fluorescent dye or another signal-emitting group.

Methods which employ combinatorial immobilized peptide libraries in conjunction with FRET have been disclosed for example in DE 19840545 A1. However, for high throughput of different substrates, there, the disadvantages of immobilized peptides are accepted—the poor accessibility of membrane-bound, densely packed substrates, the impurities in the form of incomplete synthesis products which further increase the already high background of FRET systems, and the large quantity of protease required to ensure substantially complete substrate degradation—without being able to utilize the advantages of the FRET method—the accurate analysis of enzyme kinetics.

A more recent approach which, in contrast to FRET-based techniques, was developed for longer substrates, makes use of so-called enzyme fragment complementation (Naqvi et al., 2004). For this purpose, a cyclic peptide which comprises on the one hand a cleavage site for the enzyme to be investigated, and on the other hand a peptide fragment for complementation of an otherwise inactive signal-emitting enzyme, was synthesized. Complementation is not possible in the cyclic form. The fragment suitable for complementation is only produced after cleavage of the cleavage site. However, it is necessary in this context to ensure that a cyclic peptide is cleaved just as well as a linear one, and that the complementing fragment is not attacked. These restrictions, and the relatively complicated preparation of the cyclic peptides with the appropriate purification steps, make it appear doubtful whether this method can find wide use.

With this background, the object of the present invention is to provide compounds and methods with which both the cleavability of substrates and enzyme specificities can be determined, with the disadvantages present in the prior art being overcome.

This object is achieved according to the invention in particular by the subject matter of the appended claims.

The invention in particular relates to methods for investigating the enzymatic cleavability of substrates which are characterized in that
a) compounds are provided which
   are bound to a first solid phase or are synthesized thereon;
   have a component 1 which faces the first solid phase and can be quantified;
   have a section which is to be analyzed for enzymatic cleavability;
   have a component 2 which faces away from the first solid phase and can bind directly or via a binding partner to a second solid phase;
b) the compounds are, after elimination from the first solid phase, brought into contact with an enzyme or enzyme mixture in solution;
c) the cleaved and uncleaved compounds are then bound to a second solid phase which may be identical to the first solid phase, the binding taking place via component 2, which binds directly to the second solid phase or to a binding partner put on the second solid phase;
d) the non-immobilized constituents are removed from the second solid phase;
e) the amount of uncleaved compounds is detected by quantifying component 1; and
f) the cleavability is determined by comparing the amount of uncleaved compounds before and after the cleavage reaction.

The compounds have a polymeric section which is to be analyzed for enzymatic cleavability and which is also referred to as “molecular region to be analyzed” or “substrate”. The polymeric section can be selected from the group comprising peptides, polypeptides, polysaccharides and nucleic acids. The section to be cleaved preferably takes the form of peptides.

In the context of this invention, the term peptide means an unbranched polymeric compound derived from linkage of a plurality of amino acids. An amino acid is any type of organic compound comprising at least one amine function and one acid function. The term thus not only includes natural amino acids as they occur in living creatures, but also any other synthetic compound having these properties.

In the context of this application, the term substrate (S) means the section to be analyzed for enzymatic cleavability.

The compounds of the invention thus substantially include a substrate and 2 components.

The invention further relates to methods in which component 2 can be quantified and component 1 can bind directly or via a binding partner to a second solid phase, where the binding in step c) takes place via component 1, and the uncleaved compounds are detected in step e) by quantifying component 2.

In a further embodiment of the invention, the first and second solid phase are identical.

Solid phase means any material which is suitable for direct or indirect binding of the compounds to be investigated. Inorganic materials suitable for this purpose are, for example, ceramic, silicates, glass, silicon and metals. Organic substances which can be used are for example polysaccharides such as cellulose, or polyolefins such as polystyrene, polypropylene or halogenated polyolefins (PVC, PVDF etc.). In a preferred embodiment, cellulose is used for the first solid phase and polystyrene is used for the second solid phase. In a further preferred embodiment, a transparent polystyrene microtiter plate is used as second solid phase.

The compounds provided on the solid phase include two components 1 and 2 which differ from one another, where component 1 faces the first solid phase, and component 2 faces away from the first solid phase. Small molecules are preferably chosen as components. At least one of these components must be suitable for immobilizing (in particular binding) the compound on the second solid phase. This immobilization can take place directly on the solid phase. However, it can also take place indirectly via a binding partner, for example an antibody, which is put on the solid phase. The specific immobilization usually takes place via a binding pair. The first binding partner preferably is a ligand which is smaller than the second binding partner and which, as one of the two components, is a constituent of the compound, and the second binding partner is a protein which is immobilized on the solid phase and binds the first binding partner. The covalent and noncovalent immobilization of proteins on the surface of solid phases is possible by various known methods. A covalent immobilization of the proteins can for example take place by providing carboxylate functions as reactive groups on the solid phase. Carboxylate groups can be generated on polyolefin surfaces for example by plasma oxidation or oxidation with chromic acid, permanganate or cerium sulfate. Carboxylate functions activated with carbodiimides such as, for example, N-(3-dimethylaminopropyl)-N′-ethylcarbodiimide EDC, are used to form a covalent bond to proteins. Immobilization of proteins on the surface of polystyrene microtiter plates usually takes place by noncovalent adsorption.

At least one of the components must be suitable for quantifying the compounds, i.e. it must be suitable for being able to determine the amount of compounds which is bound to the second solid phase. In the case where only one component is suitable for quantification, it must be the component which is not used for binding to the second solid phase. The component suitable for quantification may itself be signal-generating or else be a binding partner for a signal-generating group. Compounds suitable as signal-generating group are for example those capable of chemiluminescence or fluorescence. In a preferred embodiment, a high-affinity binding pair is used, where the first binding partner is a small ligand in comparison with the second binding partner and, as one of the two components, is a constituent of the compound, and the second binding partner is a protein which binds with high affinity to the first binding partner and carries a signal-emitting group. It is particularly preferred to choose biotin as first binding partner and streptavidin as second binding partner, which is conjugated to a signal-emitting group. Enzymes able to convert luminogenic, fluorogenic or chromogenic substrates are preferably used as signal-emitting group. The particularly preferred signal-emitting group is horseradish peroxidase for the catalytic conversion of colorless tetramethylbenzidine into the oxidized colored form.

In a preferred embodiment, the compounds are synthesized directly on the first solid phase, wherein it is possible to synthesize many different compounds, with different sections to be cleaved, in parallel, i.e. on different sections of the same solid phase. The substance library of peptide analytes is synthesized by methods of peptide synthesis which are known to the skilled worker. In a preferred embodiment, the FMOC synthesis method for cellulose filter-immobilized peptide libraries is used (Frank, 1992). The use of combinatorial synthesis techniques makes it possible to prepare a large number of analytes from amino acids simultaneously in parallel and location-dependently. In contrast to the FRET method, there is no experimental limitation on the length of the analyte. The length depends solely on the efficiency of the chosen synthesis method. SPOT synthesis can be used to prepare analytes with a sequence motif of at least 16 amino acids which can be chosen without restriction.

In a preferred embodiment, an anchor which can be eliminated under defined conditions and on which the desired compounds are synthesized is put on the cellulose membrane. This has the advantage that unwanted chain initiation products with carboxy-terminal truncation remain on the membrane when the compounds are eliminated. The anchor used according to the invention may consist of the amino acid units proline and lysine (Bray et al. 1990).

Following the synthesis of the anchor, component 1 is then incorporated, which terminates the compound at one end. After the sequential synthesis of the section to be cleaved, component 2 is incorporated at its other end. Herein, the components are preferably chosen in a way that they bring about minimal steric hindrance on contact of the section to be cleaved with an enzyme, and that they improve the solubility of the section to be cleaved in aqueous buffers.

In order to achieve a better solubility of the section to be cleaved and a better accessibility for the detection system, where appropriate, spacers are inserted during the course of the synthesis, preferably one spacer, between the molecular region to be analyzed and the two components, leading to the two detectable groups being spaced apart by more than 100 Å. Water-soluble spacers such as polyethylene glycol (PEG) or polyol substructures are preferably used. Polyethylene glycol substructures according to the invention mean the following structures: branched or unbranched ethylene glycol homopolymers or propylene glycol homopolymers, as well as mixed ethylene glycol/propylene glycol copolymers with average molecular weights between 100 and 5000 g/mol, substituted at one or more ends. Polyol substructures mean according to the invention linear or branched polyols which may comprise 3 to 15 hydroxyl groups.

In a preferred embodiment, chemical structures having one or more negative charges are inserted during the synthesis between the section to be cleaved and the two components, respectively. It is possible thereby substantially to prevent nonspecific binding of the compounds to the solid phases. Nonspecific binding of the compounds is an adhesion to the solid phase which is not mediated by component 1 or 2. Charge carriers within the meaning of the present invention are amino acids like those defined in the context of this invention, which carry at least one negative charge. The charges may be provided for example by phosphate, sulfate or carboxylate groups. Suitable amino acids are for example aspartic acid, glutamic acid, aminoadipic acid, carboxyaspartic acid, carboxyglutamic acid, carboxymethylcysteine, phosphoserine, phosphothreonine, phosphotyrosine, phosphonomethylphenylalanine, sulfoserine, sulfothreonine and sulfotyrosine, each in its L or D configuration. It is particularly preferred to use two D-glutamic acid units, respectively, on both sides, because alpha-peptide linkages of D-amino acids are not cleaved by proteases.

In order to avoid the negative charges influencing the cleavage reaction, in a preferred embodiment, spacers are inserted between the section to be cleaved and the negative charges on both sides. In the particularly preferred form, amino-polyethylene glycol (PEG)-diglycolic acid units each having 2 ethylene glycol units are used as spacers.

All the units mentioned which are not part of the section to be analyzed have the property of not being cleaved when the dissolved compound is brought into contact with a substance or a substance mixture whose influence on the stability of the molecular regions to be analyzed are to be established.

In one embodiment of the invention, the compounds are, after completion of the synthesis, eliminated from the first solid phase, washed out and dried in vacuo. They are then taken up in a suitable inert solvent, preferably using an aqueous, surfactant-containing buffer.

After elimination from the first solid phase, the compounds are brought into contact with an enzyme or enzyme mixture in order to assay the effects of the enzyme or enzyme mixture on the molecular region to be analyzed. In a preferred embodiment, the enzyme or enzyme mixture is selected from the group of proteases. Enzyme mixtures which can be used are purified enzymes, but also biological samples such as, for example, crude extracts of a wide variety of starting materials. Possible starting materials are intestinal fluids, stool, blood, urine, saliva, sputum, lymph fluids, other body fluids, cell lysates, tissue lysates and organ lysates.

The cleavage reaction can be terminated in various ways. Enzyme kinetics are determined by terminating after a defined time interval. In a preferred embodiment, the cleavage reaction is stopped by adding an inhibitor. In a further preferred embodiment, the cleavage reaction is terminated by decomposing the enzyme or the enzymes present in the enzyme mixture. This can take place for example by heating. The enzymatic activity can also be eliminated by changing the reaction conditions, such as, for example, changing the proton concentration and/or removing cofactors of the enzymes. The compound should not be cleaved by any of these termination methods.

Binding of the cleaved and uncleaved compounds to the second solid phase can take place via component 1. In a preferred embodiment, it takes place via component 2. This has the advantage of an additional purification step. It is possible to bind only those uncleaved analytes and cleavage products in which component 2 is completely present on the side facing away from the synthesis anchor. Analytes for which the synthesis has not proceeded to completion and cleavage products thereof, lack component 2 on the side facing away from the synthesis anchor, so that, as a consequence, they are not bound to the solid phase and detected. This is advantageous for the sensitivity of the method.

The component used for binding to the solid phase may be a partner of a high-affinity binding pair. A ligand together with a receptor forms such a binding pair, the ligand usually being a low molecular weight molecule whose stereochemical properties are matched by the receptor. When ligand and receptor meet, specific binding takes place owing to the stereochemical properties of ligand and receptor. Antibodies form a subgroup in the group of receptors. Their ligands are referred to as antigens. Small antigens unable on their own to bring about the formation of antibodies are referred to as haptens. In a preferred embodiment, the component is selected from the group of ligands or haptens. The second partner of the binding pair which is used is a solid phase-bound receptor or antibody directed against this component. In a particularly preferred embodiment, the hapten 2,4-dichlorophenoxyacetic acid (2,4-D) is used as component. In this case, the binding to the second solid phase in a preferred embodiment takes place via a monoclonal anti-2,4-D antibody (Franek, 1994) with which the second solid phase is coated. A polystyrene microtiter plate is preferably used as solid phase in this case.

In a particularly preferred embodiment, a branched or unbranched aliphatic chain is directly linked to the 2,4-D carboxylate function, thus multiply increasing the affinity of the binding between 2,4-D and antibody. In the preferred form, unbranched alkane residues with a (CH₂)ₙ chain length of n≧6 are used, the maximum chain length preferably being n=11. The alkane residue (n=11) is provided by using aminoundecanoic acid. In another preferred embodiment (n=6), the alkane residue is provided by aminohexanoic acid.

Removal of the unimmobilized constituents from the second solid phase in a preferred embodiment takes place by a washing step. In this context it is possible to employ as washing solution for example an aqueous surfactant-containing buffer such as D-PBS with Tween 20.

The amount of the uncleaved compound is determined by quantifying the component which, after the immobilization on the second solid phase, is on the side facing away from the latter. In one embodiment, this is component 2, and in a preferred embodiment, it is component 1. As already described, the component suitable for quantification may itself be signal-generating or else be a binding partner for a signal-generating group. In a preferred embodiment, the component employed for the quantification is selected from the group of ligands or haptens. AS the second partner of the binding pair a receptor or antibody directed against this component, which is coupled to a signal-emitting group, is used. It is preferred to use the ligand biotin as component and a signal-emitting group coupled to the receptor streptavidin.

The antibody with which the solid phase is coated binds the hapten, irrespective of the overall molecular structure of which the hapten forms a part. This means irrespective of whether the compound is cleaved or not cleaved. After the cleavage reaction, the solution comprises a mixture of cleaved and uncleaved compound, both of which are bound with equal affinity by the antibody. Because the cleaved compounds lack biotin, the cleaved compounds reduce the maximum signal obtained when exclusively the uncleaved compound is available for binding by the antibody. Since the antibody binds uncleaved and cleaved compounds with equal affinity, the maximum signal decreases in proportion with the content of cleaved compound. The maximum signal is determined likewise by putting the compound which has not been brought into contact with enzyme, and is accordingly uncleaved, on the coated solid phase in a separate mixture. The percentage content of cleaved compound can be determined in this way: 100*(1−signal after enzyme cleavage/signal without enzyme cleavage). If, for example, the signal after a cleavage reaction reaches 50% of the maximum signal, then half the amount of the compound employed has been cleaved. The strength of the maximum signal is determined by the amount of antibody on the solid phase.

The invention additionally relates to methods for determining enzyme kinetics, which are characterized in that the methods of the invention for investigating the enzymatic cleavability are carried out repeatedly, wherein the bringing into contact with the enzyme or enzyme mixture takes place for different time intervals or with different enzyme concentrations, and wherein the half-life of the substrates is determined.

For determination of enzyme kinetics, in one embodiment of the invention the substrate concentration [S] is generally chosen to be much higher than the concentration of the enzyme [E], so that Michaelis-Menten kinetics can be used in the evaluation. In one possible embodiment, pure enzyme solutions can be used to carry out enzymatic measurements under zero order conditions ([S]>K_{M}) which allow accurate analysis of the enzymatic mechanism of catalysis. In this embodiment it is possible to determine for a particular amount of enzyme the Michaelis-Menten constant (K_{M})—the substrate concentration at which the half-maximum enzyme rate is reached—and the maximum enzyme rate (Vₘₐₓ). Depending on the enzyme and substrate, accurately defined substrate concentrations of up to 1 mM are necessary. If this exceeds the capacity limit for parallel peptide synthesis methods such as SPOT synthesis, the compounds of the invention can also be synthesized by classical methods such as, for example, synthesis on resin supports, which is known to the skilled worker. In contrast to FRET methods, interference of substrate molecules with one another is precluded, making it possible for there to be no upper limit on the choice of substrate concentration.

In the preferred embodiment of the enzymatic cleavage, pseudo-first order conditions ([E]<[S]<K_{M}) are chosen. In a possible variation of this embodiment, several identical mixtures of enzyme and compound or substrate are brought into contact with one another for different times. The different time periods of the contact of substrate and enzyme allow the enzyme kinetics to be evaluated in analogy to a continuous measurement of the substrate conversion by chromogenic and FRET substrates. A further advantage is the fact that no accurate quantification of the amount of substrate employed is necessary for the evaluation. Detection of the cleaved and uncleaved substrate can take place with lower concentrations than in FRET methods, because a considerably lower background signal is observed from the substrate and the protease preparation employed here.

It is generally known that different substrates may vary widely in their degradation behavior. Since pseudo-first order kinetics depend exponentially on the product of measurement time and enzyme concentration, extremely short or very long measurement times may be necessary for a given enzyme concentration in order to cover the whole range of variation of degradation behavior. Since enzyme activity does not remain constant indefinitely, and there are technical limits to reducing the measurement time as desired, it is not possible to ascertain completely the whole range of substrate stability as a function of time for particular substrates.

In a particularly preferred embodiment, therefore, the enzyme concentration is varied logarithmically under pseudo-first order conditions with a given measurement time, and the degradation behavior is determined as a function of the enzyme concentration. The respective reaction rates can then be ascertained by normalization to a defined enzyme concentration.

In one possible variant of this pseudo-first order embodiment, it is possible to identify substances which have an influence on the cleavage reaction. For this purpose, the methods of the invention are carried out as described, with one or more additional measurements being carried out, which differ from the first by the addition of the substance to be tested to the reaction mixture. In a preferred embodiment, the substances are selected from the group of inhibitors consisting of competitive, tight-binding and non-competitive inhibitors. Competitive inhibitors such as, for example, the serine protease inhibitor aprotinin are particularly preferably investigated. Comparison of the efficiency of the cleavage reaction with and without inhibitor allows its activity to be ascertained.

The invention further relates to compounds which are suitable for use in the methods of the invention. Such compounds are thus suitable for investigating the enzymatic cleavability of substrates and analyzing the enzyme specificity.

The compounds of the invention have the general structure

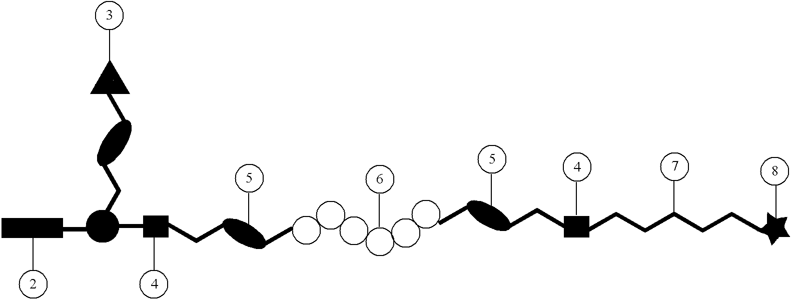

[alternatively expressed: (2)-(3)-(4)-(5)-(6)-(5)-(4)-(7)-(8)], having a component A which includes units (2) and (3) and optionally units (4) and (5), and which can be linked by unit (2) to a first solid phase and can be detected and/or quantified via unit (3), and can optionally be linked to a second solid phase, wherein the end of component A which points away from the solid phase is linked via unit (3) or, if component A includes unit (5), via unit (5) to a section (6) which is to be analyzed for enzymatic cleavability and which is linked, at its end opposite to component A, to a component B, which includes unit (8) and optionally units (4), (5) and/or (7), wherein component B is linked either directly or via unit (7) or, if component B includes unit (5), via unit (5), to the section (6) which is to be analyzed for enzymatic cleavability, and can be detected and/or quantified via unit (8) and can optionally be linked to a second solid phase, wherein
unit (2) is a synthesis anchor,
unit (3) is a detectable group,
unit (4) is a chemical structure having one or more negative charges,
unit (5) is a spacer which preferably includes polyethylene glycol or polyol substructures,
unit (7) is a spacer,
unit (8) is a detectable group,
wherein units (4) and (5) of components A and B may be identical to or different from one another, respectively.

Unit (2) preferably is a cleavable anchor via which the compound is linked to the solid phase and through selective cleavage of which it can be detached from the solid phase. Unit (2) is in particular selected from epsilon-lysyl-proline (Lys-Pro), p-[amino(2,4-dimethoxybenzyl)]phenoxyacetyl (Rink linker), p-benzyloxybenzyl alcohol (Wang linker) or 4-hydroxymethylphenoxyacetyl (HMP). In one embodiment, unit (2) is epsilon-lysyl-proline (Lys-Pro), wherein the compound is linked via the proline residue to the solid phase, and which can be cleaved. If the solid phase is cellulose, unit (2) can preferably be an amino acid. Cleavage of the resulting ester linkage can then take place by treatment with ammonia.

Unit (3) is preferably selected from the group of biotinylated amino acids. The biotinylated amino acid is in particular biocytin or N-gamma(N-biotinyl-3-(2-(2-(3-aminopropyloxy)-ethoxy)ethoxy)propyl)-L-glutamate.

In one embodiment, unit (4) consists of one or more amino acids which comprise phosphate or sulfate groups, or which are selected from the group of amino dicarboxylic acids or amino polycarboxylic acids. Examples of amino dicarboxylic acids are aspartic acid, glutamic acid, aminoadipic acid or carboxymethylcysteine, examples of polycarboxylic acids are carboxyaspartic acid or carboxylutamic acid, examples of amino acids comprising phosphate groups are phosphoserine, phosphothreonine, phosphotyrosine or phosphonomethylphenylalanine, and examples of amino acids comprising sulfate groups are sulfoserine, sulfothreonine or sulfotyrosine, each in their L or D configuration. Unit (4) preferably comprises two D-glutamic acids.

Unit (5) in components A and/or B preferably respectively comprises a polyethylene glycol substructure having a molecular mass of from 100 to 5000 g/mol or a polyol. The polyethylene glycol substructure is preferably selected from the group consisting of amino polyethylene glycol diglycolic acid with two ethylene glycol units (PEG-2, MW 530.6) and amino polyethylene glycol diglycolic acid with nine ethylene glycol units (PEG-9, MW 839.0).

Unit (7) is preferably selected from the group of aliphatic amino carboxylic acids, wherein aminoundecanoic acid or aminohexanoic acid are preferred.

Unit (8) is preferably selected from the group consisting of 2,4-dichlorophenoxyacetic acid and dinitrophenyl compounds such as, for example, 2,4-dinitrophenylglycine, 2,4-dinitrophenylaminobutyric acid, 2,4-dinitrophenylaminocaproic acid or 2,4-dinitrophenylaminoundecanoic acid.

In a particular embodiment of the invention, units (7) and (8) are combined in one unit which includes a 2,4-dichlorophenoxyacetic acid derivative of the general formula (I) which are disclosed in the international applications filed at the same time, i.e. on the same day, “Novel 2,4-dichlorophenoxyacetic acid derivatives and use thereof in diagnostic and analytic detection methods” and “Kit for highly sensitive detection assays” (applicant: in each case Forschungszentrum Borstel et al.) and in the corresponding German priority applications (DE 10 2005 051 977.6 and DE 10 2005 051 976.8), and to which express reference is hereby made.

These compounds are compounds of the formula (I)

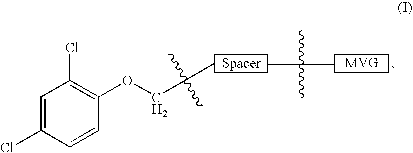

where MVG is a label-mediating group.

These compounds are stable and soluble in water, and the spacer includes 1 to 25 identical or different protected or unprotected amino acids or nucleotides. The spacer may also consist of a linear or branched chain of 1 to 10 monosaccharides, for example of glucose, mannose, galactose, ribose, arabinose, N-acetylglucosamine or fructose or be assembled from 1 to 5 disaccharide units such as cellobiose, lactose, chitobiose, lactosamine, which preferably, but not necessarily have beta-1,4-glycosidic linkages or consist of combinations or derivatives of said structures. In addition, the spacer may be assembled from O-glycosylated serine, threonine or N-glycosylated aspartic or glutamic acid subunits or comprise the latter. The spacer may additionally be assembled from linear or branched polyols having 3 to 15 hydroxyl groups which may be linked wholly or partly to the abovementioned spacer components, such as, for example, saccharide or polyol structures.

The spacer may additionally include residues selected from the following group:

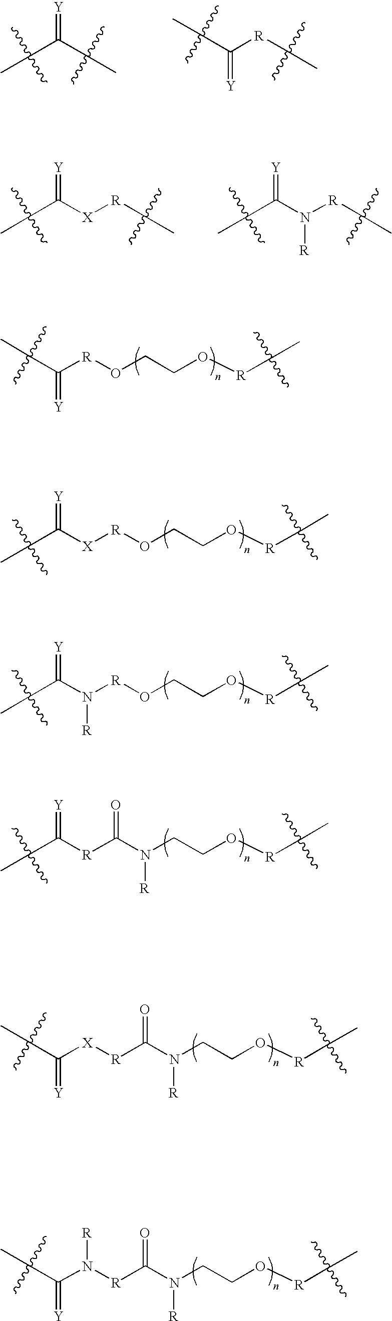

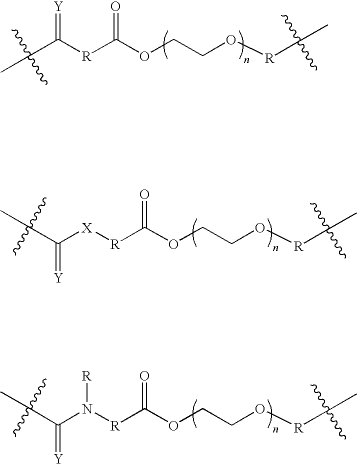

wherein X and Y are independently of one another —O— or —S—, and n is an integer in the range between 1 and 15, wherein MVG is selected from the following group:

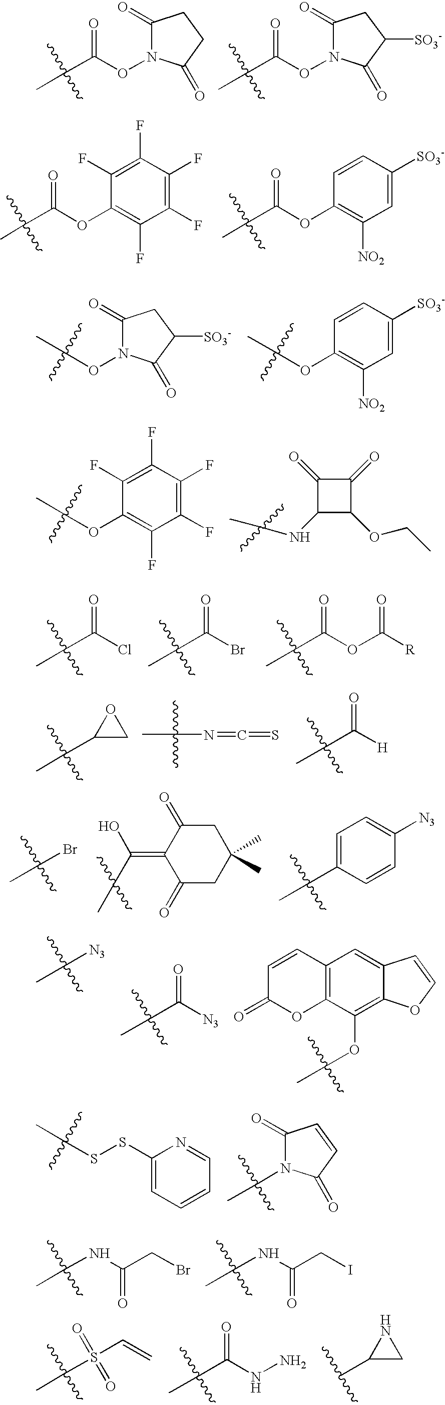

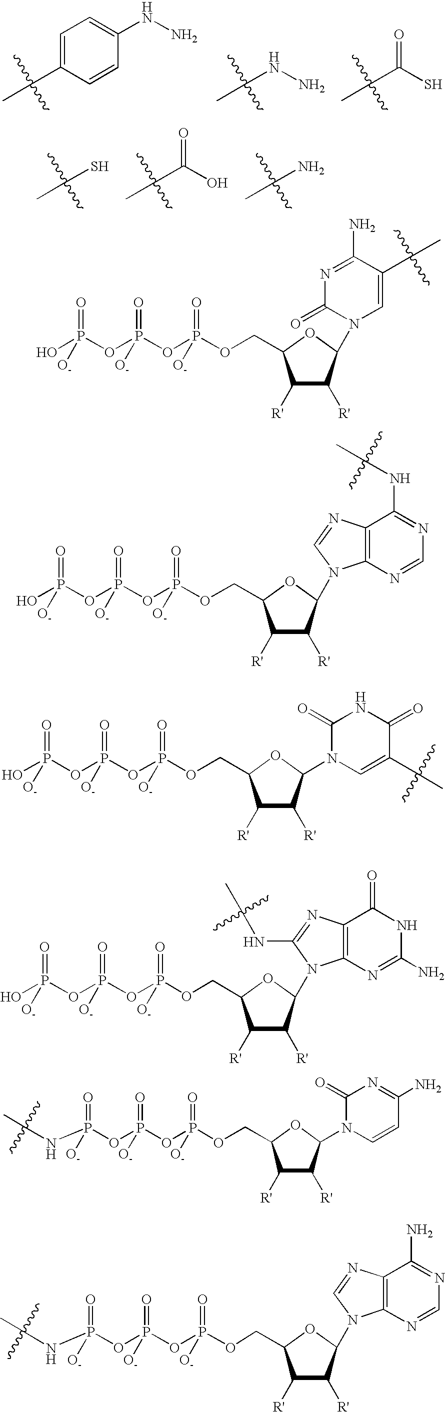

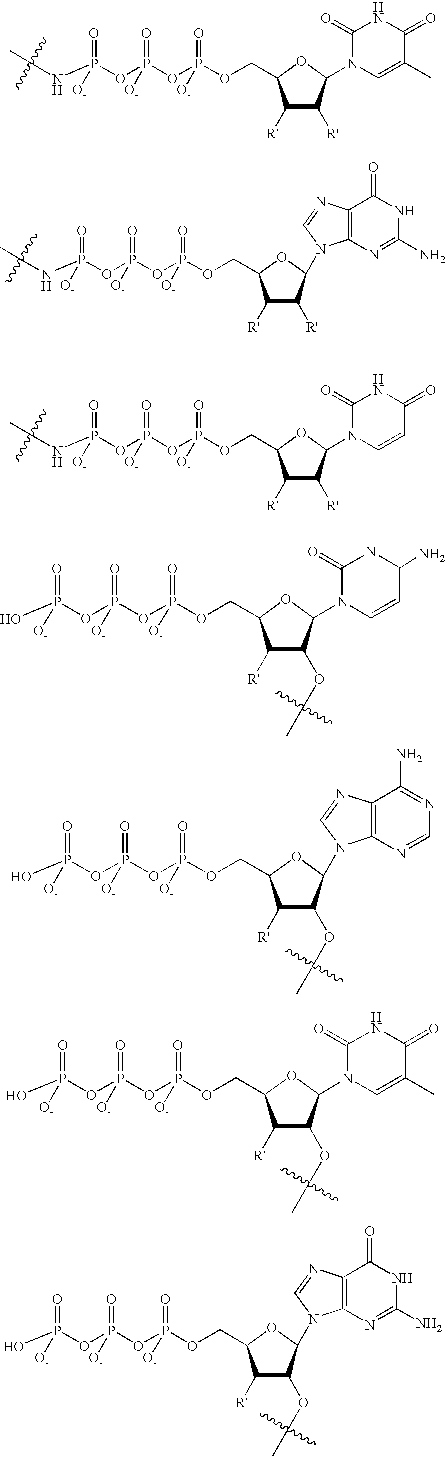

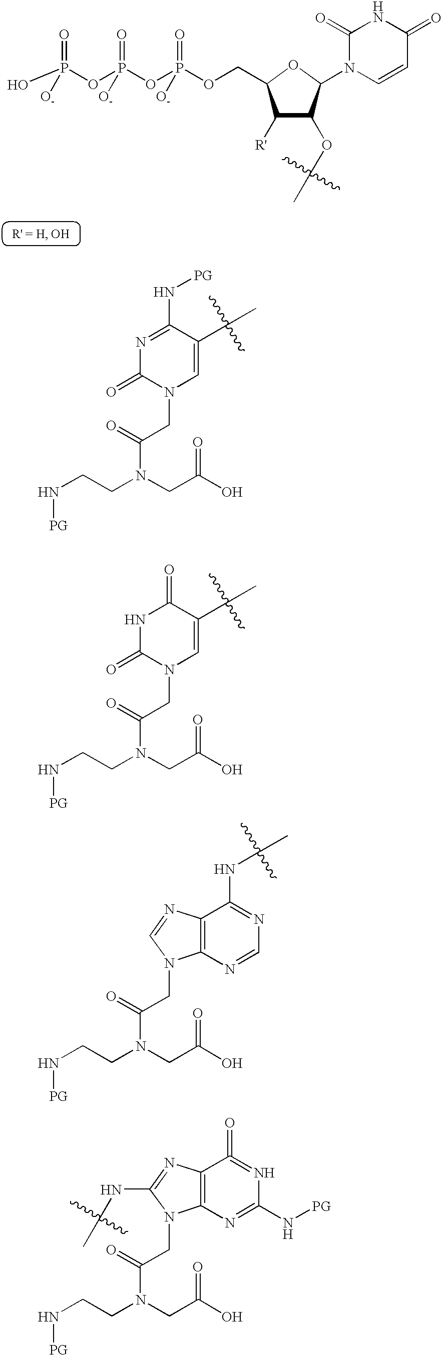

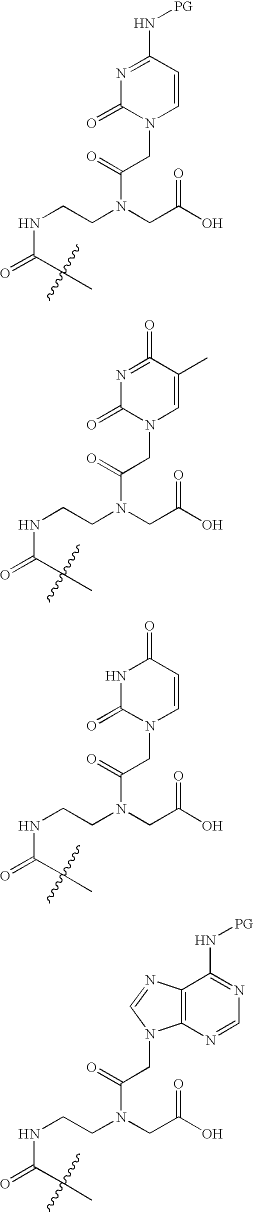

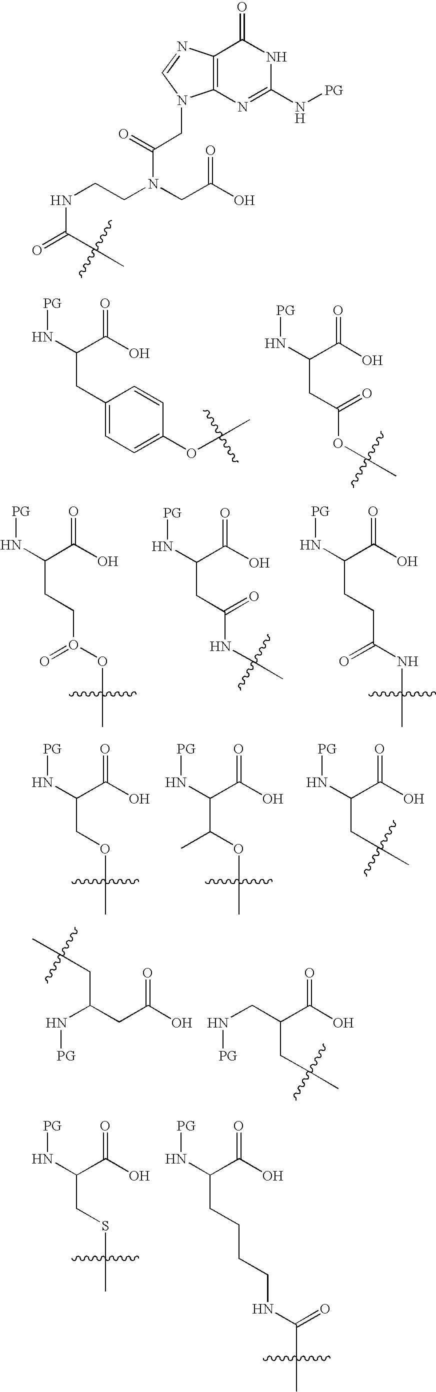

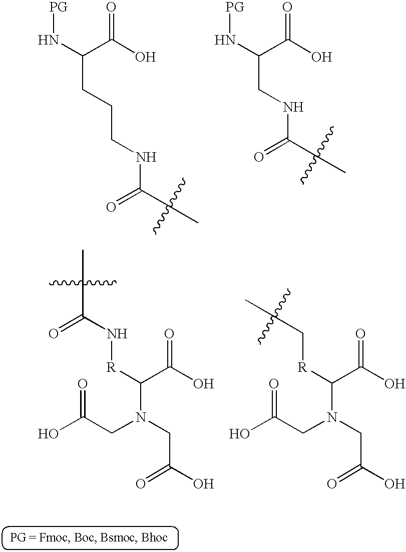

wherein R respectively are identical or different residues from the following group:
hydrogen, linear, branched or cyclic alkyl or alkoxy residue having 1 to 15 carbon atoms, linear or branched alkenyl residue having 2 to 15 carbon atoms, protected or unprotected amine.

This compound can be coupled by MVG to a section (6) which is to be analyzed for enzymatic cleavability. The coupling can take place either directly or indirectly, by coupling MVG to unit (4) or (5).

In a particular embodiment of the invention, units (4) and (5) of component A are selected from the group consisting of PEG-2/D-glutamate, PEG-2/carboxyglutamate, PEG-9/D-glutamate and PEG-9/carboxyglutamate, and independently thereof units (4) and (5) of component B are selected from the group consisting of PEG-2/D-glutamate, PEG-2/carboxyglutamate, PEG-9/D-glutamate and PEG-9/carboxyglutamate.

The invention additionally relates to kits for carrying out the methods of the invention. The kits of the invention comprise for example the compounds of the invention or components A and/or B of the compounds of the invention, solid phases, e.g. in the form of microtiter plates, the latter possibly being coated with appropriate antibodies, buffers and solutions and optionally various enzymes or also inhibitor substances.

In one embodiment of the invention, the kit comprises so-called framework constructs instead of the complete compounds of the invention.

In the present invention, a framework construct means a construct comprising the two components A and B, which optionally comprise the spacer and negative charge carriers. The framework construct thus lacks the section which is to be analyzed and is located in the middle, so that the framework construct consists of two parts which must be coupled to a section to be analyzed in order to be able to obtain the compounds of the invention and carry out the method of the invention.

The enzyme kinetics can be ascertained with the aid of the Michaelis-Menten equation. A curve-fitting program, e.g. GraphPad Prism 4 (GraphPad Software, San Diego, Calif., USA) is used to fit the function of the photometric measurements at time t and the enzyme concentration [E] by nonlinear regression to pseudo-first order reaction kinetics. The underlying formula of the exponential function for the regression can be derived from the Michaelis-Menten equation, and the following applies when [S]>>[E] is chosen for the enzymatic reaction:

v=k_{cat}[E]ₜₒₜₐₗ[S]/([S]+K_{M}) (1)

v=reaction rate
k_{cat}=catalytic constant or turnover number
[S]=substrate concentration
[E]ₜₒₜₐₗ=total enzyme concentration
K_{M}=Michaelis-Menten constant

Under pseudo-first order reaction conditions, [S] is <K_{M}. Equation (1) simplifies to:

v=k_{cat}[E]ₜₒₜₐₗ[S]/K_{M} (2)

The reaction rate v can be regarded as the change in substrate concentration with time. It is therefore possible to write (2) as a differential equation:

δ[S]ₜ/δt=−[S]ₜ[E]ₜₒₜₐₗk_{cat}/K_{M} (3)

[symbol]

[S]ₜ=[S]₀exp(−t[E]ₜₒₜₐₗk_{cat}/K_{M}) (4)

[S]ₜ=substrate concentration after time t
[S]₀=substrate concentration without contacting with the enzyme
t=time

[E]ₜₒₜₐₗk_{cat}/K_{M} corresponds to the pseudo-first order rate constant k, inserting for [E]ₜₒₜₐₗ in the preferred embodiment the enzyme concentration in the small bowel.

The photometric measurements yield values as optical density (OD) which are proportional to unconverted substrate [S]. The background signals of the measurements are taken into account in the regression to the exponential function. The following applies:

ODₜ=OD_{fin}+(OD₀−OD_{fin})exp(−t[E]ₜₒₜₐₗk_{cat}/K_{M}) (5)

ODₜ=optical density after time t
OD_{fin}=minimum attainable optical density (corresponds to the optical density limit for infinite reaction time)
OD₀=optical density without contacting with the enzyme

Since the OD is a function of t, the 3 parameters OD₀, OD_{fin} and the rate constant k (=[E]ₜₒₜₐₗk_{cat}/K_{M}) can be ascertained by determining the OD after various reaction times and by curve fitting. [E]ₜₒₜₐₗ is in this case kept constant in all measurements.

In the preferred embodiment, however, the enzyme concentration, not the time, is varied. For this purpose, several enzyme dilutions are brought into contact with in each case the same amount of substrate on a microtiter plate, and the reactions are allowed to proceed for a defined time t, in the preferred embodiment 90 minutes. The following applies:

OD_{[E]}=OD_{[E]max}+(OD₀−OD_{[E]max})exp(−t[E]ₜₒₜₐₗk_{cat}/K_{M}) (6)

OD_{[E]}=optical density with a particular enzyme concentration
OD_{[E]max}=minimum attainable optical density (corresponds to the optical density limit at infinite enzyme concentration)
OD₀=optical density without enzyme

The 3 parameters OD₀, OD_{[E]max} and the constant (tk_{cat}/K_{M}) can be determined through the dependence of the OD on the enzyme for a given time, in analogy to the dependence of the OD on time with a given enzyme concentration, by means of curve fitting.

The stability of different substrates towards a protease or protease mixture is measured in the preferred form by determining the half-life (t_{1/2}) of a substrate, since the contribution of individual enzymes to the substrate degradation cannot be accurately resolved. The following applies:

½[S]₀=[S]₀exp(−t_{1/2}[E]ₜₒₜₐₗk_{cat}/K_{M}) (7)

[symbol]

ln 0.5=t_{1/2}[E]ₜₒₜₐₗk_{cat}/K_{M} (8)

[symbol]

t_{1/2}=ln 2K_{M}/([E]ₜₒₜₐₗk_{cat}) (9)

Insertion of equation (9) into equation (5) results in:

ODₜ=OD_{fin}+(OD₀−OD_{fin})exp(−t ln(2)/t_{1/2}) (10)

[symbol]

ODₜ=OD_{fin}+(OD₀−OD_{fin})0.5̂(t/t_{1/2}) (11)

Neither the catalytic efficiency (k_{cat}/K_{M}) nor the exact enzyme concentration [E] appears in equation (11). The half-life determined using equation (11) relates to the enzyme preparation used. Since the substrate conversions measured on variation of the amount of enzyme or the incubation time (t) are proportional to one another as long as first order reaction conditions are maintained, it is possible to dilute the enzyme preparation instead of varying the incubation time, resulting in:

OD_{Df}=ODₘₐₓ+(OD₀−ODₘₐₓ)0.5̂(t*Df/t_{1/2}) (12)

Df=dilution factor for the enzyme preparation

In order to determine the efficiency of an inhibitor and the inhibition constant Kᵢ, the substrate conversion is measured both in the presence and in the absence of the inhibitor. If the inhibitor concentration employed to achieve inhibition is much higher than the enzyme concentration, the mechanism of inhibition is referred to as “classical”. It is possible in the evaluation according to Michaelis-Menten kinetics in this case to neglect the amount of inhibitor bound in the enzyme-inhibitor complex and set [I]_{free}=[I]ₜₒₜₐₗ. For a competitive inhibitor, the result is the rate equation (13):

vᵢ=k_{cat}[E]ₜₒₜₐₗ[S]/([S]+K_{M}(1+[I]ₜₒₜₐₗ/K_{i,app})) (13)

vᵢ=reaction rate of the inhibited reaction
[I]ₜₒₜₐₗ=total inhibitor concentration
K_{i,app}=apparent inhibition constant of the enzyme-inhibitor complex at a given substrate concentration; it is defined by equation (14):

K_{i,app}=Kᵢ(1+[S]/K_{M}) (14)

Kᵢ=inhibition constant

Under pseudo-first order reaction conditions ([S]<K_{M}), equations (13) and (14) result in equation (15) for the reaction rate of the inhibited reaction:

vᵢ=k_{cat}[E]ₜₒₜₐₗ[S]/K_{M}(1+[I]ₜₒₜₐₗ/Kᵢ) (15)

Equation (15) differs from equation (2) in that in the presence of an inhibitor the Michaelis Menten constant K_{M} is replaced by the extended term K_{M} (1+[I]ₜₒₜₐₗ/Kᵢ). On insertion of K_{M} (1+[I]ₜₒₜₐₗ/Kᵢ) for K_{M} in equation (9) for the inhibited reaction, the following applies analogously for the half-life:

t_{1/2,i}=ln 2K_{M}(1+[I]ₜₒₜₐₗ/Kᵢ)/([E]ₜₒₜₐₗk_{cat}) (16)

t_{1/2,i}=half-life of the inhibited reaction

To determine the inhibition constants, the following is obtained for the relationship between the reaction rates or half-lives of the inhibited and uninhibited reaction:

vᵢ/v₀=t_{1/2,i}/t_{1/2,0}=1+[I]ₜₒₜₐₗ/Kᵢ (17)

v₀=reaction rate of the uninhibited reaction
t_{1/2,0}=half-life of the uninhibited reaction

If an enzyme-catalyzed reaction is inhibited on use of equimolar amounts of enzyme and inhibitor, it follows the “tight-binding” mechanism of inhibition. This occurs when the inhibitor has high affinity or the enzyme concentration is very high. In both cases, the free inhibitor concentration is considerably reduced through the formation of the enzyme-inhibitor complex. Taking this condition into account, reaction rate equation (18) can be derived (Morrison 1969, Bieth 1995).

vᵢ/v₀=1−{[E]ₜₒₜₐₗ+[I]ₜₒₜₐₗ+K_{i,app}−{([E]ₜₒₜₐₗ+[I]ₜₒₜₐₗ+K_{i,app})²−4[E]ₜₒₜₐₗ[I]ₜₒₜₐₗ}^{0.5}}/2[E]ₜₒₜₐₗ (18)

It is possible with equation (18) to determine K_{i,app}, and to derive Kᵢ therefrom, from the measured reaction rates of the inhibited and uninhibited reaction using an iterative program, e.g. EnzFitter (Biosoft, Cambridge, UK).

The invention is described below by means of examples and figures which concern preferred embodiments of methods of the invention but do not restrict the invention.

### DESCRIPTION OF THE FIGURES

FIG. 1 shows a diagrammatic representation of a preferred embodiment of the method of the invention:

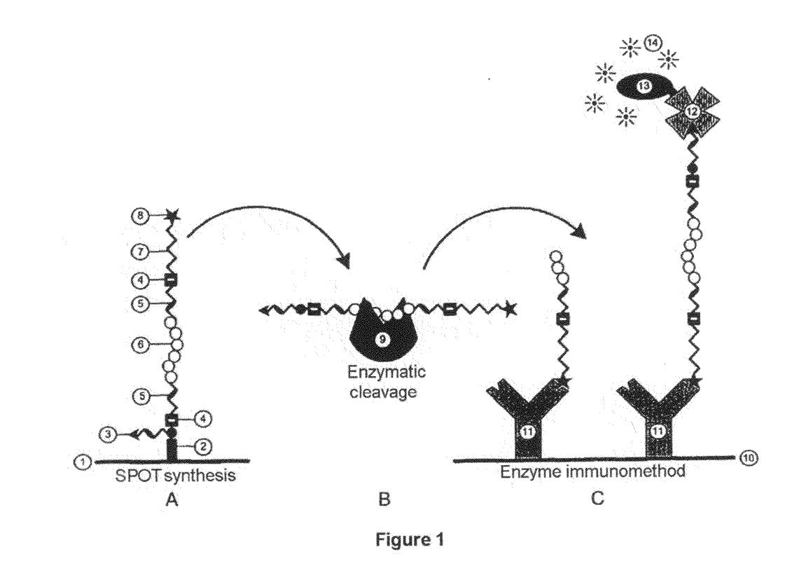

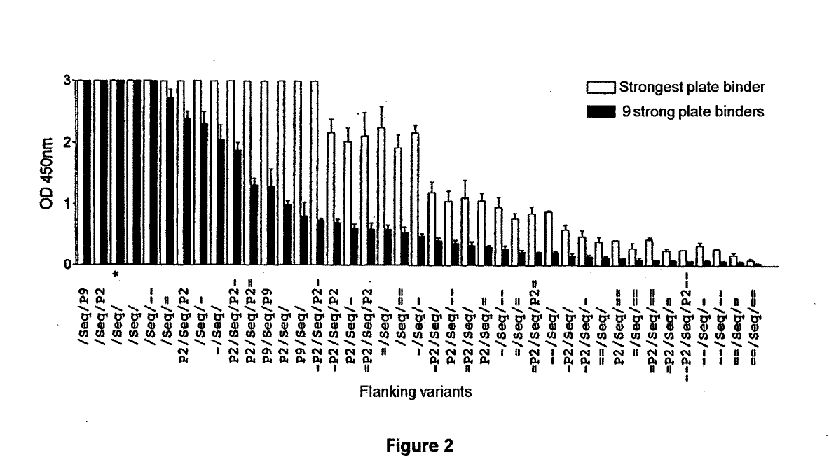

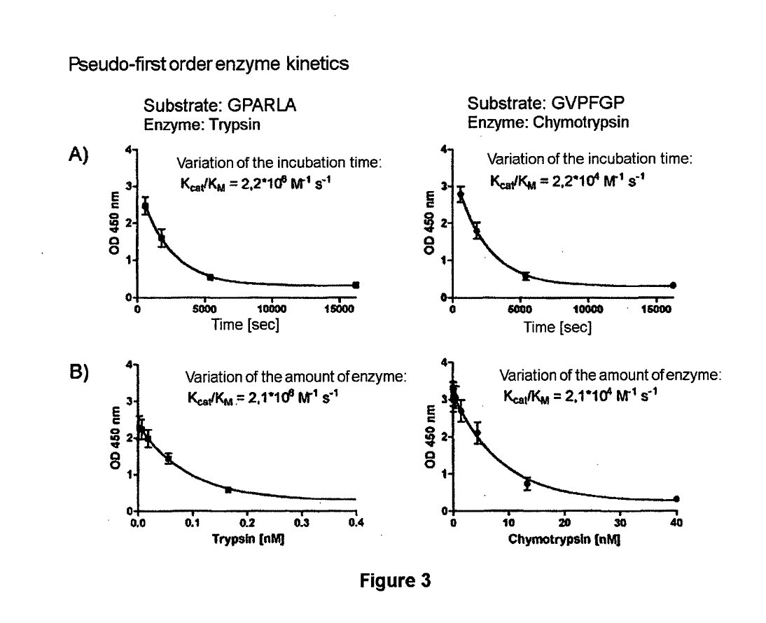

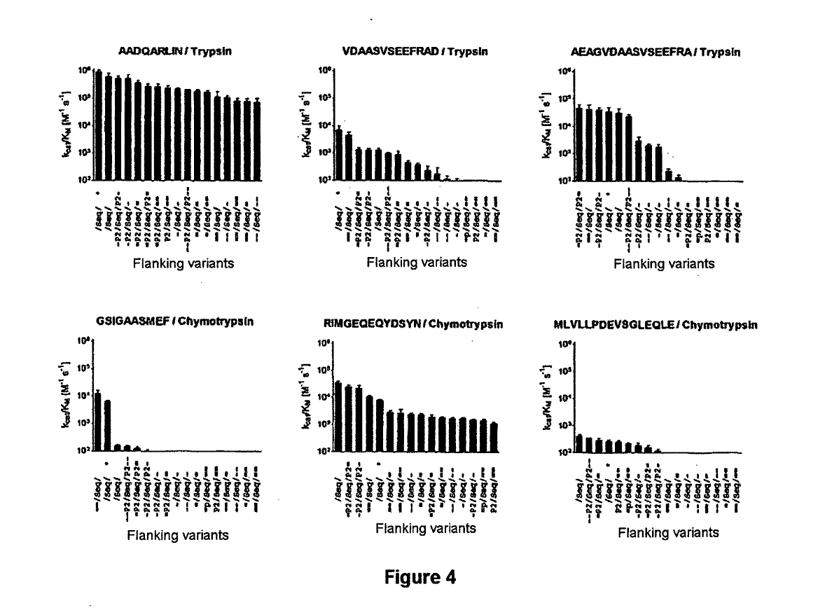

(Reference numbers: 1. cellulose membrane; 2. synthesis anchor; 3. biotin; 4. negative charges; 5. PEG spacers; 6. amino acid sequence of the molecular region to be analyzed; 7. aminoundecanoic acid spacer; 8. 2,4-dichlorophenoxyacetic acid; 9. protease; 10. microtiter plate; 11. anti-2,4-D antibody; 12. streptavidin; 13. horseradish peroxidase; 14. TMB color substrate)
(A) Oligopeptides are synthesized on a cellulose membrane (1) in the following sequence: synthesis anchor (2), biotin as detectable group or component 1 (3), carrier of a negative charge (4), PEG spacer (5), sequence motif of the molecular region of n amino acids to be analyzed (6), PEG spacer. (5), carrier of a negative charge (4), aminoundecanoic acid spacer (7) and 2,4-dichlorophenoxyacetic acid as detectable group or component 2 (8). The peptides are eliminated from the cellulose membrane after completion of the synthesis and
(B) brought into contact with a solution of proteases (9).
(C) Cleaved and uncleaved peptides are detected on a microtiter plate (10) via anti-2,4-D antibodies (11) which capture the peptide via 2,4-D (8). On the other side of the uncleaved analyte, biotin (3) binds streptavidin (12) which is coupled to the enzyme horseradish peroxidase (13). The horseradish peroxidase converts colorless TMB into the colored, oxidized form (14).

FIG. 2 shows the influence of different flanking sequences on the nonspecific binding of peptides to microtiter plates. The nine sequence motifs KIKVYLPRMK, VFKGLWEKAF, PVQMMYQIGL, VFKGLWEKAFKDE, KIKVYLPRMKMEE, FSLASRLYAEERY, ERKIKVYLPRMKMEEK, VQHFKRELMNLPQQCN, GLFRVASMASEKMKIL are distinguished by a strong tendency to bind to polystyrene microtiter plates nonspecifically. The possibility of suppressing this plate binding by providing the sequence motifs with flankings consisting of hydrophilic uncharged and/or negatively charged units was investigated. Microtiter plates were coated with an antibody which does not recognize 2,4-D, and remaining binding sites on the plate were subsequently saturated with 1% (w/v) casein in DPBS. The peptides described above with the different flankings were applied and their nonspecific binding to the microtiter plate was detected with the aid of streptavidin-coupled horseradish peroxidase and a chromogenic substrate. The diagram shows the absorptions at 450 nm obtained on use of 0.2% of the amount of a peptide of a synthesis SPOT; values ≧3 OD were determined by using 0.02% of the amount of a peptide of a synthesis SPOT and multiplied by 10. White bars show the values of the peptide which most strongly binds nonspecifically to the microtiter plate, and black bars represent the average for the nine different peptides which strongly bind nonspecifically. The different flanking regions of the peptides are identified by the following symbols: P9: amino-polyethylene glycol (PEG)-diglycolic acid with 9 ethylene glycol units; P2: amino-polyethylene glycol (PEG)-diglycolic acid with 2 ethylene glycol units; -: D-glutamate (single negative charge); carboxyglutamate (double negative charge). The carboxy-terminal label of the flanking variant identified by a * was amino-polyethylene glycol (PEG)-diglycolic acid with 9 ethylene glycol units and biocytin. All other flanking variants carried N-γ-(N-biotinyl-3-(2-(2-(3-aminopropyloxy)ethoxy)-ethoxy)-propyl)-L-glutamate as carboxy-terminal label. All peptides were provided on the amino terminus with the spacer aminoundecanoic acid and the label 2,4-D.

Introduction of negative charges into the flanking regions reduces the nonspecific plate binding more than the introduction of uncharged hydrophilic units. Moreover the extent of the reduction of plate binding depends on the number of negative charges introduced.

FIG. 3 shows the evaluation of cleavage experiments with pseudo-first order kinetics.

Degradation of the substrate GPARLA by trypsin and of GVPFGP by chymotrypsin is shown. (A) In one variation of the embodiment, the half-life was determined by varying the incubation time and (B) in the preferred embodiment by varying the enzyme concentration. [E] is kept constant in (A) (trypsin concentration: 5 ng/ml or 0.2 nM; chymotrypsin concentration: 500 ng/ml or 20 nM), and t is varied, while t is kept constant in (B) (90 min in each case) and [E] is varied. OD₀, ODₘₐₓ and t_{1/2} are each optimized as parameters on the regression curve. The catalytic efficiency (k_{cat}/K_{M}) can be calculated from t_{1/2}. The values for k_{cat}/K_{M} determined by the two methods do not differ significantly from one another (quintuplicate measurements; unpaired t test: p>0.05). In both cases, the exponential-course of a pseudo-first order reaction is clearly evident.

FIG. 4 shows the influence of different flanking sequences on the catalytic efficiency (k_{cat}/K_{M}) in the enzymatic cleavage of peptides.

In each case three different peptides whose amino acid sequence motif was flanked by different negatively charged and/or hydrophilic units were incubated with various amounts of trypsin (top of FIG. 4) or chymotrypsin (bottom of FIG. 4), and the extent of hydrolysis was determined by a subsequent enzyme immunoassay. It was possible to determine the k_{cat}/K_{M} values by nonlinear fitting of the curve of the absorptions as a function of the amount of enzyme. The designation of the flanking variants is analogous to FIG. 2.

### EXAMPLES

### Example 1

### Preparation of Oligopeptides by Spot Synthesis

Oligopeptides with a sequence length of up to 16 amino acids were synthesized by the FMOC synthesis method for cellulose membrane-immobilized peptide libraries (Frank, 1992). All the operational steps were carried out at RT. Cellulose membranes were esterified with 0.02 ml/cm² of the fluorenylmethoxycarbonyl (Fmoc)-protected amino acid proline (0.2 M Fmoc-proline, 0.46 M 1-methylimidazole and 0.26 M N,N′-diisopropylcarbodiimide (DICD) in dry N,N′-dimethylformamide (DMF)). After the coupling reaction, unreacted reactive groups were saturated with 0.13 ml/cm² 2% (v/v) acetic anhydride in DMF for 24 h. This was followed by washing with DMF three times. The Fmoc protective group was eliminated by 0.09 ml/cm² 20% (v/v) piperidine in DMF for 5 min. This was followed by washing with DMF four times. The membranes were subsequently washed with 100% ethanol three times and dried in a stream of cold air.

The following synthesis steps were carried out with an automatic pipetting machine (e.g. ASP 222, Intavis, Cologne). Firstly, a solution of 0.2 M tert-butyloxycarbonyl-(Boc)-lysine-(Fmoc)-OH and 0.35 M 1-hydroxybenzotriazole (HoBt) plus 0.25 M N,N′-diisopropylcarbodiimide (DICD) in dry, desalted 1-methyl-2-pyrrolidone (NMP) was prepared 30 min before use and incubated at RT. After a reaction time of 30 min, the solution was centrifuged in order to remove precipitates which had appeared, and 0.1 μl portions of this solution were each pipetted with the aid of the automatic pipetting machine onto defined areas of the cellulose membrane. Areas onto which the coupling solutions are pipetted are referred to as SPOTs.

After the coupling reaction, unreacted reactive groups were saturated with 0.13 ml/cm² 2% (v/v) acetic anhydride in DMF for 24 h. This was followed by washing with DMF three times. The Fmoc protective group was eliminated by 0.09 ml/cm² 20% (v/v) piperidine in DMF for 5 min. This was followed by washing with DMF five times. To detect the coupling reactions, the free amino groups on the cellulose membrane were stained with 0.13 ml/cm² of a bromophenol blue solution (0.01% (w/v) in DMF) for 10 min. The membranes were subsequently washed with 100% ethanol three times and dried in a stream of cold air.

This incubation cycle consisting of 1× acetic anhydride, 3×DMF, 1× piperidine, 5×DMF, 1× bromophenol blue and 3× ethanol was also carried out between all subsequent synthesis steps. In these cases, however, the incubation with the acetic anhydride solution, which then serves only to saturate the unreacted amino functions, was shortened to a time of 20 min. To extend the peptide chain, 0.2 μl portions of a 0.2 M amino acid active ester solution were applied for each peptide. The amino acid active ester solutions were prepared by mixing a solution of 0.2 M Fmoc-amino acid whose side chain was, if necessary, protected with suitable groups (tert-butyl (tBu) for serine, threonine, tyrosine, glutamic acid and aspartic acid; trityl for asparagine, glutamine, histidine; t-butyloxycarbonyl (Boc) for lysine and tryptophan; 2,2,4,6,7-pentamethyldihydrobenzofuran-5-sulfonyl (Pbf) for arginine and acetamidomethyl (Acm) for cysteine), and 0.35 M 1-hydroxybenzotriazole (HoBt) in dry, desalted N-methylpyrrolidinone with 1.25 mol of DICD per mol amino acid and allowing to react at RT for 30 min. Precipitates which had formed were then removed by centrifugation. The amino acid active ester solution was put on three times for each synthesis step and left to react at RT for at least 40 min each time.

The side chain protective groups (apart from Acm) were eliminated by two incubations with 0.09 ml/cm² of a solution of 50% (v/v) trifluoroacetic acid, 2% (v/v) distilled water and 3% (v/v) triisobutylsilane in dichloromethane for one hour. The cellulose-bound peptides were subsequently washed with dichloromethane four times, then with 0.1% (v/v) HCl, 50% (v/v) methanol in double-distilled water four times and finally with 1 M acetic acid, pH 1.9, four times. The membrane was dried in vacuo overnight. The SPOTs were cut out and transferred into 2 ml microreaction tubes. In order to eliminate the peptides from the membrane snippets, 500 μl of a solution of 0.1 M triethylammonium acetate (TEAA), 20% (v/v) ethanol, pH 7.5; in double-distilled water were added to each membrane snippet. They were incubated in this form overnight, and the supernatants were then put into a fresh 2 ml microreaction tube, and the elimination reaction was repeated for 2 h. The two peptide solutions were combined and the solvent was removed in vacuo. The peptides dried in this way were dissolved in 1.5 ml of 10 mM sodium phosphate buffer, pH 7.0, 10 mM NaCl (L-PBS)×0.005% (w/v) Tween 20, shock-frozen in liquid nitrogen and stored at −80° C.

The following list gives an overview of the synthesized peptides.

Sequences are indicated in the direction from the amino terminus to the carboxy terminus. The synthetic units are defined as follows: 2,4D=2,4-dichlorophenoxyacetc acid; Aun=aminoundecanoic acid; PEG9=amino-polyethylene glycol (PEG)-diglycolic acid with 9 ethylene glycol units; PEG2=amino-polyethylene glycol (PEG)-diglycolic acid with 2 ethylene glycol units; LysBio=biocytin; Anch=synthesis anchor consisting of lysine and proline; glu=D-glutamate; GluPEGBio=N-γ-(N-biotinyl-3-(2-(2-(3-aminopropyloxy)-ethoxy)-ethoxy)-propyl)-L-glutamate; Gla=L-carboxyglutamate

### 1. 2,4D-Aun-XXX-PEG9-LysBio-Anch

### 2. 2,4D-Aun-XXX-GluPEGBio-Anch

### 3. 2,4D-Aun-XXX-glu-GluPEGBio-Anch

### 4. 2,4D-Aun-glu-XXX-GluPEGBio-Anch

### 5. 2,4D-Aun-glu-XXX-glu-GluPEGBio-Anch

### 6. 2,4D-Aun-XXX-glu-glu-GluPEGBio-Anch

### 7. 2,4D-Aun-glu-XXX-glu-glu-GluPEGBio-Anch

### 8. 2,4D-Aun-glu-glu-XXX-glu-GluPEGBio-Anch

### 9. 2,4D-Aun-glu-glu-XXX-GluPEGBio-Anch

### 10. 2,4D-Aun-glu-glu-XXX-glu-glu-GluPEGBio-Anch

### 11. 2,4D-Aun-XXX-Gla-GluPEGBio-Anch

### 12. 2,4D-Aun-Gla-XXX-GluPEGBio-Anch

### 13. 2,4D-Aun-Gla-XXX-Gla-GluPEGBio-Anch

### 14. 2,4D-Aun-XXX-Gla-Gla-GluPEGBio-Anch

### 15. 2,4D-Aun-Gla-XXX-Gla-Gla-GluPEGBio-Anch

### 16. 2,4D-Aun-Gla-Gla-XXX-Gla-GluPEGBio-Anch

### 17. 2,4D-Aun-Gla-Gla-XXX-GluPEGBio-Anch

### 18. 2,4D-Aun-Gla-Gla-XXX-Gla-Gla-GluPEGBio-Anch

### 19. 2,4D-Aun-XXX-PEG2-GluPEGBio-Anch

### 20. 2,4D-Aun-PEG2-XXX-GluPEGBio-Anch

### 21. 2,4D-Aun-PEG2-XXX-PEG2-GluPEGBio-Anch

### 22. 2,4D-Aun-XXX-PEG9-GluPEGBio-Anch

### 23. 2,4D-Aun-PEG9-XXX-GluPEGBio-Anch

### 24. 2,4D-Aun-PEG9-XXX-PEG9-GluPEGBio-Anch

25. 2,4D-Aun-PEG2-XXX-PEG2-glu-GluPEGBio-Anch

### 26. 2,4D-Aun-PEG2-XXX-PEG2-Gla-GluPEGBio-Anch

### 27. 2,4D-Aun-glu-PEG2-XXX-PEG2-GluPEGBio-Anch

### 28. 2,4D-Aun-Gla-PEG2-XXX-PEG2-GluPEGBio-Anch

29. 2,4D-Aun-glu-PEG2-XXX-PEG2-glu-GluPEGBio-Anch

### 30. 2,4D-Aun-Gla-PEG2-XXX-PEG2-Gla-GluPEGBio-Anch

### 31. 2,4D-Aun-PEG2-XXX-glu-GluPEGBio-Anch

### 32. 2,4D-Aun-PEG2-XXX-glu-glu-GluPEGBio-Anch

### 33. 2,4D-Aun-PEG2-XXX-Gla-GluPEGBio-Anch

### 34. 2,4D-Aun-PEG2-XXX-Gla-Gla-GluPEGBio-Anch

### 35. 2,4D-Aun-glu-PEG2-XXX-GluPEGBio-Anch

### 36. 2,4D-Aun-glu-PEG2-XXX-glu-GluPEGBio-Anch

### 37. 2,4D-Aun-glu-PEG2-XXX-glu-glu-GluPEGBio-Anch

### 38. 2,4D-Aun-Gla-PEG2-XXX-GluPEGBio-Anch

### 39. 2,4D-Aun-Gla-PEG2-XXX-Gla-GluPEGBio-Anch

### 40. 2,4D-Aun-Gla-PEG2-XXX-Gla-Gla-GluPEGBio-Anch

41. 2,4D-Aun-glu-glu-PEG2-XXX-PEG2-glu-glu-GluPEGBio-Anch
where XXX represents the following sequence motifs (amino acids are indicated in accordance with the standard one-letter code):

a) KIKVYLPRMK
b) VFKGLWEKAF
C) PVQMMYQIGL
d) AADQARELIN
e) GSIGAASMEF
f) VFKGLWEKAFKDE
g) KIKVYLPRMKMEE
h) FSLASRLYAEERY
i) VDAASVSEEFRAD
j) RIMGEQEQYDSYN
k) ERKIKVYLPRMKMEEK
l) VQHFKRELMNLPQQCN
m) GLFRVASMASEKMKIL
n) AEAGVDAASVSEEFRA
o) MLVLLPDEVSGLEQLE

### 42. 2,4D-Aun-GPARLA-PEG9-LysBio-Anch

### 43. 2,4D-Aun-GVPFGP-PEG9-LysBio-Anch

### 44. 2,4D-Aun-GGSGPFGRSALVPEE-PEG9-LysBio-Anch

### 45. 2,4D-Aun-GGSGPDGRSALVPEE-PEG9-LysBio-Anch

### 46. 2,4D-Aun-GGSGPFGRSDLVPEE-PEG9-LysBio-Anch

### 47. 2,4D-Aun-PAPFAAA-PEG9-LysBio-Anch

### 48. 2,4D-Aun-GPARLAIG-PEG9-LysBio-Anch

and all 185 or 188 sequence motifs resulting from piecewise synthesis of the model antigen ovalbumin as peptides with a length of 16 or 10 amino acids, respectively, and a forerunner of 2 amino acids. These ovalbumin-derived peptides were synthesized with flanking regions no. 41.

Ovalbumin amino acid sequence:
GSIGAASMEFCFDVFKELKVHHANENIFYCPIAIMSALAMVYLGAKDSTR
TQINKVVRFDKLPGFGDSIEAQCGTSVNVHSSLRDILNQTKPNDVYSFSL
ASRLYAEERYPILPEYLQCVKELYRGGLEPINFQTAADQARELINSWVES
QTNGIIRNVLQPSSVDSQTAMVLVNAIVFKGLWEKAFKDEDTQAMPFRVT
EQESKPVQMMYQIGLFRVASMASEKMKILELPFASGTMSMLVLLPDEVSG
LEQLESIINFEKLTEWTSSNVMEERKIKVYLPRMKMEEKYNLTSVLMAMG
ITDVFSSSANLSGISSAESLKISQAVHAAHAEINEAGREVVGSAEAGVDA
ASVSEEFRADHPFLFCIKHIATNAVLFFGRCVSP

### Example 2

### Obtaining an Intestinal Enzyme Solution from the Small Bowel of Mice by Intestinal Lavage

Anesthetized female Balb/c mice were sacrificed by cervical dislocation and their abdominal wall was opened. The small bowel was clamped with a hemostat behind the stomach outlet (proximal end) and severed from the stomach. The small bowel was exposed and clamped with a second hemostat near the posterior (distal) end. The bowel was severed a second time behind this point.

The subsequent preparation steps to obtain the digestive secretions were performed on an ice-cooled surface. The small bowel closed on both sides was agitated in ice-cold simulated intestinal fluid (SIF; 8 mM phosphate, pH 7.2, with 4.6 mM K⁺, 111.3 mM Na⁺, 101.5 mM Cl) according to Lockwood and Randall (1949), which was intended to imitate as accurately as possible the physiological conditions of the small bowel in terms of ion concentrations, in order to remove external contaminants and keep it moist. To obtain the digestive secretions present in the small bowel, the distal end of the bowel was suspended in a reaction vessel and the hemostat attached on this side was removed. A lateral cut was then made at the proximal end of the bowel so that a syringe provided with a button cannula could be introduced into the bowel which was held without tension. The bowel contents were washed out in three steps. The washing solutions were successively injected slowly into the bowel and collected at the lower end of the bowel separately in three reaction vessels. Firstly, the bowel was rinsed with a volume of 6 ml of perfluorohexane, an inert liquid which is immiscible with water, and the perfluorohexane was driven out with 4 ml of air. Then a mixture of 2.25 ml of perfluorohexane and 0.25 ml of SIF which contained the nonphysiological ions Li⁺ and Cs⁺, was used for washing in order to be able subsequently to determine the dilution by spectroscopic methods. Both ions have no influence on the protease activity of intestinal lavage. In a third washing step, 5 ml of SIF without tracer were injected. Solid constituents of the bowel contents and the perfluorohexane were separated by centrifugation from the aqueous phase, in which the digestive enzymes were present. An inductively coupled plasma mass spectrometry (ICP-MS) was used to determine the concentrations of Li⁺ and Cs⁺ in the intestinal enzyme solution, and the dilution factor was calculated taking the volume of the washing buffer into account. The dilution of the third lavage was ascertained via endogenous markers of intestinal secretion (e.g. enzymic activities, immunoglobulins), in comparison with the first two intestinal enzyme solutions. The enzyme solutions were stored after freezing with liquid nitrogen at −70° C.

### Example 3

### Investigation of the Influence of Hydrophilic Groups and Charges on the Nonspecific Binding Behavior of Peptides on Microtiter Plates

The following peptides mentioned in example 1 were used to analyze the nonspecific binding on the microtiter plates: framework construct 1 to 41 in combination with sequence motifs a-c, f-h, k-m. High-bind microtiter plates with 96 (8×12) wells (e.g. from Costar/Corning, Wiesbaden) were used for the enzyme immunomethod. The plates were coated by pipetting 75 μl of a freshly prepared solution of 50 ng/ml polyclonal mouse IgG which had no specificity for 2,4-D in L-PBS into each individual well. After incubation at 4° C. overnight, the coating solution was aspirated out, and the wells of the microtiter plate were washed three times with a surfactant-containing buffer (DPBST) (Dulbecco's PBS (DPBS): 2.7 mM KCl, 1.5 mM KH₂PO₄, 136 mM NaCl, 8.1 mM Na₂HPO₄, pH 7.3; DBST: DPBS×0.05% (w/v) Tween 20) with the aid of an automatic plate washer (e.g. Columbus Washer, Tecan, Crailsheim) and then sucked empty. In order to reduce nonspecific deposits on the microtiter plate as far as possible, its wells were filled with a saturation solution (1% (w/v) casein in DPBS). After renewed incubation at RT for at least three hours, the saturation solution was removed by suction as before, and the microtiter plate was washed four times with DPBST. The wells which had been sucked empty were filled with peptide solution. For this purpose, the peptide solutions obtained after the peptide synthesis in example 1 were diluted 1:10 in L-PBS×0.005% (w/v) Tween 20. For each peptide, one well of a microtiter plate was filled with 45 μl, and another one was filled with 72 μl, of SIF×0.005% (w/v) Tween 20. Then 30 μl of the respective diluted peptide solution were put into the first well, and 3 μl into the second. 75 μl of SIF×0.005% (w/v) Tween 20 only were put into 6 wells of each microtiter plate to determine a background signal. After an incubation time of 2.5 h at RT, the unbound constituents of the mixture were removed by aspiration and washing four times with DPBST, and the wells were sucked empty. Peptides bound to the plate were provided with peroxidase as reporter enzyme by adding 75 μl of 1 μg/ml peroxidase-labeled streptavidin in DPBS with 1% (w/v) casein. After one hour, the streptavidin solution was aspirated off and the microtiter plate was washed six times with DPBST and sucked empty. The color signal was developed by adding in each case 75 μl of a tetramethylbenzidine color substrate to each well of the microtiter plate in the dark and, after 30 min, development was stopped by adding 125 μl of 1 M sulfuric acid to each. The color signal was determined by photometry at a wavelength of 450 nm using a microtiter plate reader (e.g. VersaMax, Molecular Devices, Ismaning).

FIG. 2 shows the differences in the strength of nonspecific plate binding obtained with various peptides as a function of the different flanking regions.

### Example 4

### Coating and Saturation of the Wells of Microtiter Plates for Subsequent Loading with 2,4-D-Labeled Peptides

High-bind microtiter plates with 96 (8×12) wells (e.g. from Costar/Corning, Wiesbaden) were used for the enzyme immunomethod. The plates were coated by pipetting 75 μl of a freshly prepared solution of 50 ng/ml anti-2,4-D antibody (Franek et al., 1994) in L-PBS into each individual well. After incubation at 4° C. overnight, the coating solution was aspirated off, and the wells of the microtiter plate were washed three times with a surfactant-containing buffer (DPBST) (Dulbecco's PBS (DPBS): 2.7 mM KCl, 1.5 mM KH₂PO₄, 136 mM NaCl, 8.1 mM Na₂HPO₄, pH 7.3; DBST: DPBS×0.05% (w/v) Tween 20) with the aid of an automatic plate washer (e.g. Columbus Washer, Tecan, Crailsheim) and then sucked empty. In order to prevent nonspecific deposits on the microtiter plate, its wells were filled with a saturation solution (1% (w/v) casein in DPBS). After incubation at RT again for at least three hours, the saturation solution was aspirated off as before, and the microtiter plate was washed four times with DPBST and the wells were sucked empty.

### Example 5

### Enzymatic Conversion of Substrates with a Defined Trypsin and Chymotrypsin Enzyme Solution

On a polypropylene microtiter plate having the same format as the coated polystyrene microtiter plate, 7.5 μl of peptide solution were introduced into the first well of a row, and 5 μl of the peptide solution were introduced into each of the next 10 wells. The peptide chosen for the trypsin enzyme had the amino acid sequence GPARLA (number 42 from example 1) and the peptide chosen for the chymotrypsin enzyme had the sequence GVPFGP (number 43 from example 1). The last well remained empty and was intended to serve as background correction. Then, simulated intestinal fluid (SIF) with the addition of 0.005% Tween 20 and 1 mM CaCl (SIFT-CaCl) was put in all the wells. The well with 7.5 μl of peptide solution received 60 μl of SIFT-CaCl, and the wells with 5 μl of peptide solution each received 45 μl of SIFT-CaCl. The empty well for the background correction received 50 μl of SIFT-CaCl. The enzymatic reaction was then started by adding 7.5 μl of enzyme solution (10 μg/ml trypsin or chymotrypsin solution) to the well with 7.5 μl of peptide solution and the appropriate volume of SIFT-CaCl. 25 μl of this mixture were immediately pipetted into the next well and mixed, and this step was repeated to result in a serial 1:3 dilution of the enzyme solution over a total of 10 wells. No enzyme solution was put in the last peptide-containing well. Each well thus contained a volume of 50 μl after the serial dilution. The polypropylene plate was covered with a dimpled lid and incubated at 37° C. for 90 min. The enzymatic reaction was then stopped by adding 50 μl of a protease inhibitor solution (308 nM aprotinin, 20 μM leupeptin, 400 μM 4-(2-aminoethyl)benzenesulfonyl fluoride (AEBSF) hydrochloride in L-PBS) to each well, and the microtiter plate was covered with a dimpled lid. After 10 min at 4° C., the microtiter plate was heated at 90° C. for 10 min and cooled at 0° C. for at least 5 min before further use.

### Example 6

### Inhibition of an Enzymatic Conversion

Inhibition constants were measured by carrying out two parallel enzyme dilutions in each case. One enzyme dilution was carried out in the presence, and the other enzyme dilution in the absence, of the protease inhibitor aprotinin. On a polypropylene microtiter plate having the same format as the coated polystyrene microtiter plate, 7.5 μl of peptide solution were introduced into the first well of a row, and 5 μl of the peptide solution were introduced into each of the next 10 wells. The peptide chosen for the trypsin enzyme had the amino acid sequence GPARLA (number 42 from example 1) and the peptide chosen for the chymotrypsin enzyme had the sequence GVPFGP (number 43 from example 1). The last well remained empty and was intended to serve as background correction. Then, to carry out the proteolytic cleavage in the presence of an inhibitor, 7.5 μl of an aprotinin solution (1 μg/ml for trypsin and 1 mg/ml for chymotrypsin) were put into the first well, and 5 μl of the aprotinin solution (1 μg/ml for trypsin and 1 mg/ml for chymotrypsin) were put into each of the next 10 wells. The last well remained empty and was intended to serve as background correction. Then simulated intestinal fluid (SIF) with the addition of 0.005% (w/v) Tween 20 and 1 mM CaCl (SIFT-CaCl) was put into all the wells. The well with 7.5 μl of peptide solution without aprotinin solution received 60 μl of SIFT-CaCl and the wells with 5 μl of peptide solution without aprotinin solution each received 451 μl of SIFT-CaCl. The well with 7.5 μl of peptide solution and 7.5 μl of aprotinin solution received 52.5 μl of SIFT-CaCl and the wells with 5 μl of peptide solution and 5 μl of aprotinin solution each received 40 μl of SIFT-CaCl. The empty well for the background correction received 50 μl of SIFT-CaCl. The enzymatic reaction was then started by adding 7.5 μl of enzyme solution (10 μg/ml trypsin or 1 mg/ml chymotrypsin solution) to the well with 7.5 μl of peptide solution and 60 μl of SIF-CaCl or 52.5 μl of SIF-CaCl and 7.5 μl of aprotinin solution. 25 μl of this mixture were immediately pipetted into the next well and mixed, and this step was repeated to result in a serial 1:3 dilution of the enzyme solution over a total of 10 wells. No enzyme solution was put in the last peptide-containing well. Thus, each well contained a volume of 50 μl after the serial dilution. The polypropylene plate was covered with a dimpled lid and incubated at 37° C. for 90 min. The enzymatic reaction was then stopped by adding 50 μl of a protease inhibitor solution (308 nM aprotinin, 20 μM leupeptin, 400 μM 4-(2-aminoethyl)benzenesulfonyl fluoride (AEBSF) hydrochloride in L-PBS) to each well, and the microtiter plate was covered with a dimpled lid. After 10 min at 4° C., the microtiter plate was heated at 90° C. for 10 min and cooled at 0° C. for at least 5 min before further use.

### Example 7

### Investigation of the Influence of Hydrophilic Groups in the Flanking Sequences on the Enzymatic Cleavability of Peptides

On a polypropylene microtiter plate having the same format as the coated polystyrene microtiter plate, 7.5 μl of peptide solution were introduced into the first well of a row, and 5 μl of the peptide solution were introduced into each of the next 10 wells. The peptides used for the investigations with the enzyme trypsin were the following ones detailed in example 1: framework construct 1 to 41 in combination with sequence motifs d, i and n. The peptides used for the investigations with the enzyme chymotrypsin were the following ones detailed in example 1: framework construct 1 to 41 in combination with sequence motifs e and o and framework construct 1 to 40 in combination with sequence motif j. The last well remained empty and was intended to serve as background correction. Then, simulated intestinal fluid (SIF) with the addition of 0.005% Tween 20 and 1 mM CaCl (SIFT-CaCl) was put into all the wells. The well with 7.5 μl of peptide solution received 60 μl of SIFT-CaCl, and the wells with 5 μl of peptide solution each received 45 μl of SIFT-CaCl. The empty well for the background correction received 50 μl of SIFT-CaCl. The enzymatic reaction was then started by adding 7.5 μl of enzyme solution (500 μg/ml trypsin or chymotrypsin solution) to the well with 7.5 μl of peptide solution and 60 μl of SIFT-CaCl. 25 μl of this mixture were immediately pipetted into the next well and mixed, and this step was repeated to result in a serial 1:3 dilution of the enzyme solution over a total of 10 wells. No enzyme solution was put in the last peptide-containing well. Each well thus contained a volume of 50 μl after the serial dilution. The polypropylene plate was covered with a dimpled lid and incubated at 37° C. for 90 min. The enzymatic reaction was then stopped by adding 50 μl of a protease inhibitor solution (308 nM aprotinin, 20 μM leupeptin, 400 μM 4-(2-aminoethyl)benzenesulfonyl fluoride (AEBSF) hydrochloride in L-PBS) to each well, and the microtiter plate was covered with a dimpled lid. After 10 min at 4° C., the microtiter plate was heated at 90° C. for 10 min and cooled at 0° C. for at least 5 min before further use.

### Example 8

### Enzymatic Conversion of Substrates with an Intestinal Enzyme Solution

On a polypropylene microtiter plate having the same format as the coated polystyrene microtiter plate (see example 4), 7.5 μl of peptide solution were introduced into the first well of a row, and 5 μl of the peptide solution were introduced into each of the next 10 wells. The last well remained empty and was intended to serve as background correction. Then, simulated intestinal fluid (SIF) with the addition of 0.005% Tween 20 and 1 mM CaCl (SIFT-CaCl) was put in all the wells. The well with 7.5 μl of peptide solution received 60 μl of SIFT-CaCl, and the wells with 5 μl of peptide solution each received 45 μl of SIFT-CaCl. The empty well for the background correction received 50 μl of SIFT-CaCl. The enzymatic reaction was then started by adding 7.5 μl of a dilution of an intestinal enzyme solution (obtained as in example 2) to the well with 7.5 μl of peptide solution and 60 μl of SIFT-CaCl. 25 μl of this mixture were immediately pipetted into the next well and mixed, and this step was repeated to result in a serial 1:3 dilution of the intestinal enzyme solution over a total of 10 wells. No enzyme solution was put into the last peptide-containing well. Each well thus contained a volume of 50 μl after the serial dilution. The polypropylene plate was covered with a dimpled lid and incubated at 37° C. for 90 min. The enzymatic reaction was then stopped by adding 50 μl of a protease inhibitor solution (308 nM aprotinin, 20 μM leupeptin, 400 μM 4-(2-aminoethyl)benzenesulfonyl fluoride (AEBSF) hydrochloride in L-PBS) to each well, and the microtiter plate was covered with a dimpled lid. After 10 min at 4° C., the microtiter plate was heated at 90° C. for 10 min and cooled at 0° C. for at least 5 min before further use.

### Example 9

### Detection of Uncleaved Peptides after Binding to a Solid Phase

A multichannel pipette was used to put 75 μl each of the reaction mixture from example 5, 6, 7 or 8 onto the microtiter plate coated with anti-2,4-D antibody and saturated (see example 4 for the coating). After an incubation time of 2.5 h at RT, the unbound constituents of the reaction mixture were removed by aspiration and washing four times with DPBST, and the wells were sucked empty. The uncleaved compounds were provided with peroxidase as reporter enzyme by adding 75 μl of 1 μg/ml peroxidase-labeled streptavidin in DPBS with 1% (w/v) casein. After one hour, the solution was aspirated off and the microtiter plate was washed six times with DPBST and sucked empty. The color signal was developed by adding 75 μl portions of a tetramethylbenzidine color substrate to each well of the microtiter plate. The development took place in the dark and was stopped after 30 min by adding 125 μl of 1 M sulfuric acid, respectively. The color signal was determined by photometry at a wavelength of 450 nm using a microtiter plate reader (e.g. VersaMax, Molecular Devices, Ismaning).

The photometric measurements of a serially diluted reaction mixture with protease and of the reaction mixture without enzyme were used for nonlinear curve fitting by using previously described equation (6):

OD_{[E]}=ODₘₐₓ+(OD₀−ODₘₐₓ)exp(−t[E]ₜₒₜₐₗk_{cat}/K_{M})

to determine the three parameters OD₀, ODₘₐₓ and catalytic efficiency (k_{cat}/K_{M}) for experiments with the defined trypsin and chymotrypsin solutions and the appropriate substrates. The catalytic efficiency is a measure of the effectiveness of cleavage of a substrate by an enzyme.

GPRARLA (number 42 from example 1) was cleaved with trypsin with a catalytic efficiency of 2.0*10⁶±0.9*10⁶ M⁻¹s⁻¹, and GVPFGP (number 43 from example 1) was cleaved with chymotrypsin with a catalytic efficiency of 2.3*10⁴±0.9*10⁴ M⁻¹s⁻¹ (in each case geometric mean and standard deviation of a quintuplicate measurement). For comparison, the GPARLAIG variant of the trypsin substrate was cleaved with trypsin with a catalytic efficiency of 1.2*10⁶ M⁻¹s⁻¹ with FRET-based methods (Grahn et al., 1998).

The inhibition constant of aprotinin can be calculated by comparing the catalytic efficiency (k_{cat}/K_{M}) in the presence and in the absence of aprotinin. k_{cat}/K_{M} values are calculated according to equation (6) and the relationship obtained (equation (17)):

(k_{cat}/K_{M})₀/(k_{cat}/K_{M})=1+[I]ₜₒₜₐₗ/Kᵢ

The enzymatic degradation of the substrate GPARLA (number 42 from example 1) by trypsin was inhibited by aprotinin with an inhibition constant of 1.0*10⁻⁹+9.1*10⁻¹⁰ M and the degradation of GVPFGP (number 43 from example 1) by chymotrypsin was inhibited by aprotinin with an inhibition constant of 4.8*10⁻⁷±8.1*10⁻⁸ M (in each case geometric mean and standard deviation of a quadruplicate measurement).

Analysis of the influence of hydrophilic and negatively charged units in the flanking regions on the enzymatic hydrolysis revealed that the presence of negative charges immediately following the substrate sequence in most cases led to a deterioration in the catalytic efficiency compared with substrates without adjacent negative charges. It was possible in almost all cases to eliminate this interfering effect of the negative charges again by incorporating a hydrophilic PEG spacer between substrate sequence and negative charge. This is depicted in FIG. 4.

### Example 10

### Determination of the Half-Lives of Peptide Epitopes from a Model Antigen in Intestinal Fluid

To verify the applicability of the method to a large number of widely different peptides, the half-lives (t_{1/2}) of 186 16mer and of 188 10mer peptides, each of which cover the complete amino acid sequence of the model antigen ovalbumin with overlap, in an intestinal enzyme solution were determined. The peptides were synthesized as described in example 1. In this case, the peptides were provided with the amino-terminal flanking sequence 2,4D-Aun-glu-glu-PEG2- and with the carboxy-terminal flanking sequence -PEG2-glu-glu-GluPEGBio (flanking sequences 41 in example 1). The proteolytic cleavage of the peptides was carried out as described in example 8 in dilute murine intestinal enzyme solution (obtained as in example 2) and the remaining amount of uncleaved peptides was then determined as described in example 9.

The half-life of the individual peptides in undiluted intestinal fluid was calculated by using the photometric measurements (OD_{D}) at various dilutions of the intestinal enzyme solution (Df) for a nonlinear curve fitting to equation (12):

OD_{D}=ODₘₐₓ+(OD₀−ODₘₐₓ) O,5̂(5400 sec*Df/t_{1/2}).

The half-lives obtained in this way are shown in table 1. A range of half-lives in undiluted intestinal fluid from 0.00084 sec to 40.13 sec was covered in this example.

TABLE 1

Half-lives of 10 mer and 16 mer peptides
from the model antigen ovalbumin in
murine intestinal fluid.

| 10-mer peptides | | 16-mer peptides | |
|---|---|---|---|
| Sequence | t_{1/2} [sec] | Sequence | t_{1/2} [sec] |

| | | | |
|---|---|---|---|
| GSIGAASMEF | 2.7013 | GSIGAASMEFCFDVFK | 0.0097 |
| IGAASMEFCF | 0.0629 | IGAASMEFCFDVFKEL | 0.0084 |
| AASMEFCFDV | 0.0556 | AASMEFCFDVFKELKV | 0.0068 |
| SMEFCFDVFK | 0.0103 | SMEFCFDVFKELKVHH | 0.0084 |
| EFCFDVFKEL | 0.0088 | EFCFDVFKELKVHHAN | 0.0098 |
| CFDVFKELKV | 0.0122 | CFDVFKELKVHHANEN | 0.0103 |
| DVFKELKVHH | 0.0101 | DVFKELKVHHANENIF | 0.0201 |
| FKELKVHHAN | 0.0094 | FKELKVHHANENIFYC | 0.0074 |
| ELKVHHANEN | 0.1969 | ELKVHHANENIFYCPI | 0.0851 |
| KVHHANENIF | 1.2897 | KVHHANENIFYCPIAI | 0.0892 |
| HHANENIFYC | 0.0485 | HHANENIFYCPIAIMS | 0.0575 |
| ANENIFYCPI | 0.1557 | ANENIFYCPIAIMSAL | 0.0425 |
| ENIFYCPIAI | 0.0890 | ENIFYCPIAIMSALAM | 0.0327 |
| IFYCPIAIMS | 0.0469 | IFYCPIAIMSALAMVY | 0.0167 |
| YCPIAIMSAL | 0.2911 | YCPIAIMSALAMVYLG | 0.0125 |
| PIAIMSALAM | 0.1596 | PIAIMSALAMVYLGAK | 0.0077 |
| AIMSALAMVY | 0.0512 | AIMSALAMVYLGAKDS | 0.0088 |
| MSALAMVYLG | 0.0132 | MSALAMVYLGAKDSTR | 0.0063 |
| ALAMVYLGAK | 0.0077 | ALAMVYLGAKDSTRTQ | 0.0045 |
| AMVYLGAKDS | 0.0111 | AMVYLGAKDSTRTQIN | 0.0037 |
| VYLGAKDSTR | 0.0135 | VYLGAKDSTRTQINKV | 0.0040 |
| LGAKDSTRTQ | 0.0122 | LGAKDSTRTQINKVVR | 0.0033 |
| AKDSTRTQIN | 0.0092 | AKDSTRTQINKVVRFD | 0.0038 |
| DSTRTQINKV | 0.0210 | DSTRTQINKVVRFDKL | 0.0044 |
| TRTQINKVVR | 0.0027 | TRTQINKVVRFDKLPG | 0.0045 |
| TQINKVVRFD | 0.0094 | TQINKVVRFDKLPGFG | 0.0085 |
| INKVVRFDKL | 0.0069 | INKVVRFDKLPGFGDS | 0.0111 |
| KVVRFDKLPG | 0.0119 | KVVRFDKLPGFGDSIE | 0.0131 |
| VRFDKLPGFG | 0.1526 | VRFDKLPGFGDSIEAQ | 0.1209 |
| FDKLPGFGDS | 25.2450 | FDKLPGFGDSIEAQCG | 5.6930 |
| KLPGFGDSIE | 40.1300 | KLPGFGDSIEAQCGTS | 6.4211 |
| PGFGDSIEAQ | 17.8110 | PGFGDSIEAQCGTSVN | 5.0348 |
| FGDSIEAQCG | 7.1220 | FGDSIEAQCGTSVNVH | 1.3951 |
| DSIEAQCGTS | 13.4820 | DSIEAQCGTSVNVHSS | 0.4688 |
| IEAQCGTSVN | 6.4814 | IEAQCGTSVNVHSSLR | 0.1930 |
| AQCGTSVNVH | 4.3435 | AQCGTSVNVHSSLRDI | 0.0267 |
| CGTSVNVHSS | 1.1378 | CGTSVNVHSSLRDILN | 0.0499 |
| TSVNVHSSLR | 0.1177 | TSVNVHSSLRDILNQI | 0.1029 |
| VNVHSSLRDI | 0.0148 | VNVHSSLRDILNQITK | 0.0614 |
| VHSSLRDILN | 0.0527 | VHSSLRDILNQITKPN | 0.1104 |
| SSLRDILNQI | 0.1504 | SSLRDILNQITKPNDV | 0.1963 |
| LRDILNQITK | 0.0939 | LRDILNQITKPNDVYS | 0.0514 |
| DILNQITKPN | 4.1593 | DILNQITKPNDVYSFS | 0.0223 |
| LNQITKPNDV | 20.7000 | LNQITKPNDVYSFSLA | 0.0054 |
| QITKPNDVYS | 0.2963 | QITKPNDVYSFSLASR | 0.0025 |
| TKPNDVYSFS | 0.0120 | TKPNDVYSFSLASRLY | 0.0011 |
| PNDVYSFSLA | 0.0026 | PNDVYSFSLASRLYAE | 0.0014 |
| DVYSFSLASR | 0.0037 | DVYSFSLASRLYAEER | 0.0011 |
| YSFSLASRLY | 0.0019 | YSFSLASRLYAEERYP | 0.0032 |
| FSLASRLYAE | 0.0035 | FSLASRLYAEERYPIL | 0.0031 |
| LASRLYAEER | 0.0031 | LASRLYAEERYPILPE | 0.0023 |
| SRLYAEERYP | 0.0052 | SRLYAEERYPILPEYL | 0.0028 |
| LYAEERYPIL | 0.1052 | LYAEERYPILPEYLQC | 0.0281 |
| AEERYPILPE | 11.5000 | AEERYPILPEYLQCVK | 0.2682 |
| ERYPILPEYL | 1.466. | ERYPILPEYLQCVKEL | 0.1018 |
| YPILPEYLQC | 0.1016 | YPILPEYLQCVKELYR | 0.0298 |
| ILPEYLQCVK | 0.3817 | ILPEYLQCVKELYRGG | 0.0143 |
| PEYLQCVKEL | 0.1101 | PEYLQCVKELYRGGLE | 0.0152 |
| YLQCVKELYR | 0.0263 | YLQCVKELYRGGLEPI | 0.0093 |
| QCVKELYRGG | 0.0128 | QCVKELYRGGLEPINF | 0.0085 |
| VKELYRGGLE | 0.0225 | VKELYRGGLEPINFQT | 0.0052 |
| ELYRGGLEPI | 0.0244 | ELYRGGLEPINFQTAA | 0.0085 |
| YRGGLEPINF | 0.0238 | YRGGLEPINFQTAADQ | 0.0110 |
| GGLEPINFQT | 0.0336 | GGLEPINFQTAADQAR | 0.0207 |
| LEPINFQTAA | 0.0242 | LEPINFQTAADQAREL | 0.0155 |
| PINFQTAADQ | 0.0812 | PINFQTAADQARELIN | 0.0183 |
| NFQTAADQAR | 0.1293 | NFQTAADQARELINSW | 0.0247 |
| QTAADQAREL | 0.0514 | QTAADQARELINSWVE | 0.0297 |
| AADQARELIN | 0.0325 | AADQARELINSWVESQ | 0.0322 |
| DQARELINSW | 0.0224 | DQARELINSWVESQTN | 0.0223 |
| ARELINSWVE | 0.0156 | ARELINSWVESQTNGI | 0.0078 |
| ELINSWVESQ | 1.2344 | ELINSWVESQTNGIIR | 0.1455 |
| INSWVESQTN | 3.3348 | INSWVESQTNGIIRNV | 0.0095 |
| SWVESQTNGI | 0.7564 | SWVESQTNGIIRNVLQ | 0.0099 |
| VESQTNGIIR | 0.3309 | VESQTNGIIRNVLQPS | 0.0085 |
| SQTNGIIRNV | 0.0066 | SQTNGIIRNVLQPSSV | 0.0032 |
| TNGIIRNVLQ | 0.0072 | TNGIIRNVLQPSSVDS | 0.0033 |
| GIIRNVLQPS | 0.0038 | GIIRNVLQPSSVDSQT | 0.0050 |
| IRNVLQPSSV | 0.0049 | IRNVLQPSSVDSQTAM | 0.0069 |
| NVLQPSSVDS | 0.3384 | NVLQPSSVDSQTAMVL | 0.1618 |
| LQPSSVDSQT | 0.1866 | LQPSSVDSQTAMVLVN | 0.0332 |
| PSSVDSQTAM | 4.5647 | PSSVDSQTAMVLVNAI | 0.0371 |
| SVDSQTAMVL | 0.2013 | SVDSQTANVLVNAIVF | 0.0236 |
| DSQTAMVLVN | 0.0195 | DSQTAMVLVNAIVFKG | 0.0080 |
| QTAMVLVNAI | 0.0264 | QTAMVLVNAIVFKGLW | 0.0051 |
| AMVLVNAIVF | 0.0165 | AMVLVNAIVFKGLWEK | 0.0061 |
| VLVNAIVFKG | 0.0087 | VLVNAIVFKGLWEKAF | 0.0030 |
| VNAIVFKGLW | 0.0051 | VNAIVFKGLWEKAFKD | 0.0028 |
| AIVFKGLWEK | 0.0054 | AIVFKGLWEKAFKDED | 0.0037 |
| VFKGLWEKAF | 0.0039 | VFKGLWEKAFKDEDTQ | 0.0057 |
| KGLWEKAFKD | 0.0053 | KGLWEKAFKDEDTQAM | 0.0084 |
| LWEKAFKDED | 0.0131 | LWEKAFKDEDTQAMPF | 0.0101 |
| EKAFKDEDTQ | 0.0329 | EKAFKDEDTQAMPFRV | 0.0037 |
| AFKDEDTQAM | 7.6573 | AFKDEDTQAMPFRVTE | 0.0047 |
| KDEDTQAMPF | 0.6468 | KDEDTQAMPFRVTEQE | 0.0049 |
| EDTQAMPFRV | 0.0033 | EDTQAMPFRVTEQESK | 0.0041 |
| TQAMPFRVTE | 0.0047 | TQAMPFRVTEQESKPV | 0.0076 |
| AMPFRVTEQE | 0.0063 | AMPFRVTEQESKPVQM | 0.0050 |
| PFRVTEQESK | 0.1310 | PFRVTEQESKPVQMMY | 0.1097 |
| RVTEQESKPV | 2.1948 | RVTEQESKPVQMMYQI | 0.1086 |
| TEQESKPVQM | 19.4780 | TEQESKPVQMMYQIGL | 0.0696 |
| QESKPVQMMY | 3.8079 | QESKPVQMMYQIGLFR | 0.0055 |
| SKPVQMIAYQI | 0.1240 | SKPVQMMYQIGLFRVA | 0.0020 |
| PVQMMYQIGL | 0.0880 | PVQMMYQIGLFRVASM | 0.0022 |
| QMMYQIGLFR | 0.0060 | QMMYQIGLFRVASMAS | 0.0021 |
| MYQIGLFRVA | 0.0025 | MYQIGLFRVASMASEK | 0.0025 |
| QIGLFRVASM | 0.0015 | QIGLFRVASMASEKMK | 0.0021 |
| GLFRVASMAS | 0.0060 | GLFRVASMASEKMKIL | 0.0047 |
| FRVASMASEK | 0.0082 | FRVASMASEKMKILEL | 0.0026 |
| VASMASEKMK | 0.1745 | VASMASEKMKILELPF | 0.0075 |
| SMASEKMKIL | 0.0072 | SMASEKMKILELPFAS | 0.0073 |
| ASEKMKILEL | 0.0103 | ASEKMKILELPFASGT | 0.0034 |
| EKMKILELPF | 0.0135 | EKMKILELPFASGTMS | 0.0134 |
| MKILELPFAS | 0.0183 | MKILELPFASGTMSML | 0.0220 |
| ILELPFASGT | 0.0767 | ILELPFASGTMSMLVL | 0.0507 |
| ELPFASGTMS | 0.0648 | ELPFASGTMSMLVLLP | 0.0410 |
| PFASGTMSML | 3.8440 | PFASGTMSMLVLLPDE | 0.0444 |
| ASGTMSMLVL | 0.1692 | ASGTMSMLVLLPDEVS | 0.0325 |
| GTMSMLVLLP | 0.0949 | GTMSMLVLLPDEVSGL | 0.0306 |
| MSMLVLLPDE | 0.0317 | MSMLVLLPDEVSGLEQ | 0.0139 |
| MLVLLPDEVS | 0.3230 | MLVLLPDEVSGLEQLE | 0.1049 |
| VLLPDEVSGL | 8.3587 | VLLPDEVSGLEQLESI | 0.2344 |
| LPDEVSGLEQ | 0.3722 | LPDEVSGLEQLESIIN | 0.1597 |
| DEVSGLEQLE | 0.1719 | DEVSGLEQLESIINFE | 0.0265 |
| VSGLEQLESI | 1.0014 | VSGLEQLESIINFEKL | 0.0393 |
| GLEQLESIIN | 0.5162 | GLEQLESIINFEKLTE | 0.0228 |
| EQLESIINFE | 0.0686 | EQLESIINFEKLTEWT | 0.0345 |
| LESIINFEKL | 0.0663 | LESIINFEKLTEWTSS | 0.0424 |
| SIINFEKLTE | 0.0667 | SIINFEKLTEWTSSNV | 0.0653 |
| INFEKLTEWT | 0.0541 | INFEKLTEWTSSNVME | 0.0497 |
| FEKLTEWTSS | 0.0563 | FEKLTEWTSSNVMEER | 0.0286 |
| KLTEWTSSNV | 0.4176 | KLTEWTSSNVMEERKI | 0.0414 |
| TEWTSSNVME | 0.5842 | TEWTSSNVMEERKIKV | 0.0139 |
| WTSSNVMEER | 0.9286 | WTSSNVMEERKIKVYL | 0.0060 |
| SSNVMEERKI | 0.0399 | SSNVMEERKIKVYLPR | 0.0021 |
| NVMEERKIKV | 0.0072 | NVMEERKIKVYLPRMK | 0.0021 |
| MEERKIKVYL | 0.0037 | MEERKIKVYLPRMKME | 0.0014 |
| ERKIKVYLPR | 0.0008 | ERKIKVYLPRMKMEEK | 0.0012 |
| KIKVYLPRMK | 0.0017 | KIKVYLPRNKMEEKYN | 0.0016 |
| KVYLPRMKME | 0.0044 | KVYLPRMKMEEKYNLT | 0.0030 |
| YLPRMKMEEK | 0.0088 | YLPRMKMEEKYNLTSV | 0.0054 |
| PRMKMEEKYN | 0.0056 | PRMKMEEKYNLTSVLM | 0.0051 |
| MKMEEKYNLT | 0.0367 | MKMEEKYNLTSVLMAM | 0.0321 |
| MEEKYNLTSV | 0.0093 | MEEKYNLTSVLMAMGI | 0.0225 |
| EKYNLTSVLM | 0.0124 | EKYNLTSVLMAMGITD | 0.0211 |
| YNLTSVLMAM | 0.0294 | YNLTSVLMAMGITDVF | 0.0298 |
| LTSVLMAMGI | 0.0787 | LTSVLMAMGITDVFSS | 0.0170 |
| SVLMAMGITD | 0.1194 | SVLMAMGITDVFSSSA | 0.0276 |
| LMAMGITDVF | 0.8333 | LMAMGITDVFSSSANL | 0.0241 |
| AMGITDVFSS | 0.0154 | AMGITDVFSSSANLSG | 0.0262 |
| GITDVFSSSA | 0.0178 | GITDVFSSSANLSGIS | 0.0240 |
| TDVFSSSANL | 0.0190 | TDVFSSSANLSGISSA | 0.0361 |
| VFSSSANLSG | 0.1849 | VFSSSANLSGISSAES | 0.2471 |
| SSSANLSGIS | 0.7012 | SSSANLSGISSAESLK | 0.3682 |
| SANLSGISSA | 0.6149 | SANLSGISSAESLKIS | 0.0601 |
| NLSGISSAES | 1.5216 | NLSGISSAESLKISQA | 0.0601 |
| SGISSAESLK | 0.5455 | SGISSAESLKISQAVH | 0.0526 |
| ISSAESLKIS | 0.0401 | ISSAESLKISQAVHAA | 0.0502 |
| SAESLKISQA | 0.0451 | SAESLKISQAVHAAHA | 0.0475 |
| ESLKISQAVH | 0.0465 | ESLKISQAVHAAHAEI | 0.0463 |
| LKISQAVHAA | 0.0192 | LKISQAVHAAHAEINE | 0.0154 |
| ISQAVHAAHA | 0.1691 | ISQAVHAAHAEINEAG | 0.1789 |
| QAVHAAHAEI | 0.1777 | QAVHAAHAEINEAGRE | 0.0901 |
| VHAAHAEINE | 0.1356 | VHAAHAEINEAGREVV | 0.0783 |
| AAHAEINEAG | 0.7772 | AAHAEINEAGREVVGS | 0.1269 |
| HAEINEAGRE | 0.1388 | HAEINEAGREVVGSAE | 0.1531 |
| EINEAGREVV | 0.1025 | EINEAGREVVGSAEAG | 0.1950 |
| NEAGREVVGS | 0.1284 | NEAGREVVGSAEAGVD | 0.1676 |
| AGREVVGSAE | 0.0967 | AGREVVGSAEAGVDAA | 0.1212 |
| REVVGSAEAG | 1.7169 | REVVGSAEAGVDAASV | 0.0694 |
| VVGSAEAGVD | 4.0618 | VVGSAEAGVDAASVSE | 0.0475 |
| GSAEAGVDAA | 3.1271 | GSAEAGVDAASVSEEF | 0.0261 |
| AEAGVDAASV | 0.0711 | AEAGVDAASVSEEFRA | 0.0214 |
| AGVDAASVSE | 0.0548 | AGVDAASVSEEFRADH | 0.0322 |
| VDAASVSEEF | 0.0426 | VDAASVSEEFRADHPF | 0.0389 |
| AASVSEEFRA | 0.0246 | AASVSEEFRADHPFLF | 0.0233 |
| SVSEEFRADH | 0.0788 | SVSEEFRADHPFLFCI | 0.0029 |
| SEEFRADHPF | 0.0759 | SEEFRADHPFLFCIKH | 0.0038 |
| EFRADHPFLF | 0.0569 | EFRADHPFLFCIKHIA | 0.0031 |
| RADHPFLFCI | 0.0056 | RADHPFLFCIKHIATN | 0.0038 |
| DHPFLFCIKH | 0.0042 | DHPFLFCIKHIATNAV | 0.0046 |
| PFLFCIKHIA | 0.0046 | PFLFCIKHIATNAVLF | 0.0055 |
| LFCIKHIATN | 0.0185 | LFCIKHIATNAVLFFG | 0.0038 |
| CIKHIATNAV | 0.0929 | CIKHIATNAVLFFGRC | 0.0025 |
| KHIATNAVLF | 0.0719 | KHIATNAVLFFGRCVS | 0.0008 |
| IATNAVLFFG | 0.0048 | HIATNAVLFFGRCVSP | 0.0009 |
| TNAVLFFGRC | 0.0015 | | |
| AVLFFGRCVS | 0.0012 | | |
| VLFFGRCVSP | 0.0014 | | |

The model antigen ovalbumin was synthesized in the form of 188 overlapping 10mer peptides and 186 overlapping 16mer peptides (in each case 2 amino acids forerunner) on cellulose membrane and provided during the synthesis with the amino-terminal flanking sequence 2,4D-Aun-glu-glu-PEG2- and with the carboxy-terminal flanking sequence -PEG2-glu-glu-GluPEGBio. After elimination from the membrane, the peptides were incubated in various dilutions of an enzyme solution isolated from murine small bowel for 90 min. The peptides were then immobilized on polystyrene plates coated with anti-2,4-D antibodies, and the remaining amount of peptides which had not been cleaved proteolytically was determined by detecting the carboxy-terminal biotin with the aid of peroxidase-coupled streptavidin and a colorogenic substrate. The photometric measurements with various dilutions of the intestinal enzyme solution (OD_{D}) were inserted in the equation OD_{D}=ODₘₐₓ+(OD₀−ODₘₐₓ) O,5̂(5400 sec*Df/t_{1/2}), where Df is the respective dilution factor of the enzyme solution, and the half-lives t_{1/2} were derived therefrom by nonlinear curve fitting. The indicated half lives relate to undiluted intestinal fluid.

### REFERENCES

Bender M et al. The Determination of the Concentration of Hydrolytic Enzyme Solutions: α-Chymotrypsin, Trypsin, Papain, Elastase, Subtilisin, and Acetylcholinesterase. 1966; J. Am. Chem. Soc. 88:5890-5913.
Bieth J. Theoretical and Practical Aspects of Proteinase Inhibition Kinetics. 1995; Methods Enzymol. 248:59-84.
Bray et al. The simultaneous multiple production of solution phase peptides: assessment of the Geysen method of simultaneous peptide synthesis. 1990; Tetrahedron Letters 31(40):5811-5814.
Coombs G et al. Distinct Mechanisms Contribute to Stringent Substrate Specificity of Tissue-type Plasminogen Activator. 1996; J. Biol. Chem. 271:4461-4467.
Franek M et al. Monoclonal ELISA for 2,4-Dichlorphenoxyacetic Acid: Characterization of Antibodies and Assay Optimization. 1994; J. Agric. Food Chem. 42:1369-1374.
Frank R. Spot synthesis: an easy technique for the positionally addressable, parallel chemical synthesis on a membrane support. 1992; Tetrahedron 48:9217-9232.
George J et al. Evaluation of an Imaging Platform during the Development of a FRET Protease Assay. 2003; J. Biomol. Screen. 8:72-80.
Grahn, S et al. Design and synthesis of fluorogenic trypsin peptide substrates based on resonance energy transfer. 1998; Anal. Biochem. 265: 225-231.
Haber E et al. Application of a radioimmunoassay for angiotensin I to the physiologic measurements of plasma renin activity in normal human subjects. 1969; J. Clin. Endocrin. Metab. 29:1349-1355.
Latt S et al. Fluorescence determination of carboxypeptidase A activity based on electronic energy transfer. 1972; Anal. Biochem. 50:56-62.
Lockwood J, Randall H A T. The place of electrolyte studies in surgical patients. 1949 Bull. N.Y. Acad. Med. 24:228
Morrison J. Kinetics of the reversible inhibition of enzyme catalysed reactions of tight-binding inhibitors. 1969; Biochim. Biophys. Acta 185:269-265.
Naqvi et al. β-Galaktosidase Enzyme Fragment Complementation as a High-Throughput Screening Protease Technology. 2004; J. Biomol. Screen. 9:398-408.
O. Gutiérrez et al. An immunoenzymatic solid-phase assay for quantitative determination of HIV-1 protease acitivity. 2002; Anal. Biochem. 307:18-24
Wang et al. A Continuous Fluorescence Assay of Renin Activity. 1993; Anal. Biochem. 210:351-359.
Yaron A et al. Intramolecularly Quenched Fluorogenic Substrates for Hydrolytic Enzymes. 1979; Anal. Biochem. 95:228-235.

1. A method for investigating the enzymatic cleavability of substrates, characterized in that
a) compounds are provided which
are bound to a first solid phase or are synthesized thereon;
have a component 1 which faces the first solid phase and can be quantified;
have a section which is to be analyzed for enzymatic cleavability;
have a component 2 which faces away from the first solid phase and can bind directly or via a binding partner to a second solid phase;
b) after elimination from the first solid phase, the compounds are brought into contact with an enzyme or enzyme mixture in solution;
c) the cleaved and uncleaved compounds are then bound to a second solid phase which may be identical to the first solid phase, the binding taking place via component 2 which binds directly to the second solid phase or to a binding partner put on the second solid phase;
d) the unimmobilized constituents are removed from the second solid phase;
e) the amount of uncleaved compounds is detected by quantifying component 1; and
f) the cleavability is determined by comparing the amount of uncleaved compounds before and after the cleavage reaction.

2. The method of claim 1, characterized in that component 2 can be quantified and component 1 can bind directly or via a binding partner to a second solid phase, wherein the binding in step c) takes place via component 1, and the uncleaved compounds are detected in step e) by quantifying component 2.

3. A method for determining enzyme kinetics, characterized in that methods of claim 1 are carried out repeatedly, wherein the contact in step b) takes place for different time intervals or with different enzyme concentrations, and the half-life of the substrates is determined.

4. The method of claim 1, characterized in that in step d) the unimmobilized constituents are removed from the second solid phase by a washing step.

5. The method of claim 1, characterized in that the first and the second solid phase are identical.

6. The method of claim 1, characterized in that the polymeric section to be analyzed is a peptide.

7. The method of claim 1, characterized in that component 1 and 2 are different and are selected from the group comprising ligands, haptens and biotin, respectively.

8. The method of claim 7, characterized in that the hapten is 2,4-dichlorophenoxyacetic acid (2,4-D).

9. The method of claim 1, characterized in that the first and the second solid phase consist of a material which is selected from the group comprising silicates, ceramic, glass, metal, organic substances.

10. The method of claim 1, characterized in that substrates with different peptide sections are prepared in parallel from amino acids.

11. The method of claim 1, characterized in that the enzyme or enzyme mixture is selected from the group of proteases.

12. The method of claim 1, characterized in that in step b) an inhibitor whose influence on the cleavage reaction is to be analyzed is additionally added.

13. A compound of the general structure
(2)-(3)-(4)-(5)-(6)-(5)-(4)-(7)-(8),
which has a component A comprising units (2) and (3) and optionally units (4) and (5) and which can be linked by unit (2) to a first solid phase and can be detected and/or quantified via unit (3) and can optionally be linked to a second solid phase, wherein the end of component A which points away from the solid phase is linked via unit (3) or, if component A includes unit (5), via unit (5) to a section (6) which is to be analyzed for enzymatic cleavability, and which is linked, at its end opposite to component A, to a component B comprising unit (8) and optionally units (4), (5) and/or (7), where component B is linked either directly or via unit (7) or, if component B includes unit (5), via unit (5) to the section (6) which is to be analyzed for enzymatic cleavability, and can be detected and/or quantified via unit (8) and can optionally be linked to a second solid phase, wherein
unit (2) is a synthesis anchor,
unit (3) is a detectable group,
unit (4) is a chemical structure having one or more negative charges,
unit (5) is a spacer,
unit (7) is a spacer,
unit (8) is a detectable group,
wherein units (4) and (5) of components A and B may be identical to or different from one another, respectively.

14. The compound of claim 13, characterized in that unit (2) is a cleavable anchor, through selective cleavage of which the compound can be detached from the solid phase.

15. The compound of claim 13, characterized in that unit (2) is selected from epsilon-lysyl-proline (Lys-Pro), p-[amino (2,4-dimethoxybenzyl)]phenoxyacetyl (Rink linker), p-benzyloxybenzyl alcohol (Wang linker) or 4-hydroxymethylphenOxyacetyl (HMP).

16. The compound of claim 15, characterized in that unit (2) is epsilon-lysyl-proline (Lys-Pro), wherein the compound can be linked to the solid phase via the proline residue.

17. The compound of claim 13, characterized in that unit (3) is selected from the group of biotinylated amino acids.

18. The compound of claim 17, wherein the biotinylated amino acid is biocytin or N-gamma(N-biotinyl-3-(2-(2-(3-aminopropyloxy)ethoxy)ethoxy)propyl) L-glutamate.

19. The compound of claim 13, characterized in that unit (5) in each case includes a polyethylene glycol structure having a molecular mass of from 100 to 5000 g/mol or a polyol.

20. The compound of claim 19, characterized in that the polyethylene glycol structure is selected from the group consisting of amino polyethylene glycol diglycolic acid with two ethylene glycol units (PEG-2, MW 530.6) and amino polyethylene glycol diglycolic acid with nine ethylene glycol units (PEG-9, MW 839.0).

21. The compound of claim 13, characterized in that unit (4) consists of one or more amino acids which comprise phosphate or sulfate groups, or from the group of amino dicarboxylic acids or amino polycarboxylic acids.

22. The compound of claim 21, characterized in that the amino acids of unit (4) are selected from the group consisting of glutamate, carboxyglutamate, aspartate and aminoadipic acid.

23. The compound of claim 13, characterized in that unit (7) is selected from the group of aliphatic amino carboxylic acids.

24. The compound of claim 23, characterized in that unit (7) is aminoundecanoic acid or aminohexanoic acid.

25. The compound of claim 13, characterized in that unit (8) is selected from the group consisting of 2,4-dichlorophenoxyacetyl and dinitrophenyl compounds.

26. The compound of claim 13, characterized in that units (7) and (8) are combined in one unit, which includes a 2,4-dichlorophenoxyacetic acid derivative of the general formula (I) of the German patent application “Neue 2,4-Dichlorphenoxyessigsäurederivate und deren Verwendung in diagnostischen und analytischen Nachweisverfahren” (applicant: Forschungszentrum Borstel), filed simultaneously.

27. The compound of claim 13, characterized in that units (4) and (5) of component A are selected from the group consisting of PEG-2/D-glutamate, PEG-2/carboxy-glutamate, PEG-9/D-glutamate and PEG-9/carboxyglutamate, and independently thereof units (4) and (5) of component B are selected from the group consisting of PEG-2/D-gluta-mate, PEG-2/carboxyglutamate, PEG-9/D-glutamate and PEG-9/carboxyglutamate.

28. A kit for carrying out the methods of claim 1, characterized in that it comprises components A and B of the compounds, solid phases, buffers and solutions and, optionally, various enzymes or also inhibitor substances.

29. A kit for carrying out the methods of claim 1, characterized in that it comprises the compounds, solid phases, buffers and solutions and optionally various enzymes or also inhibitor substances.
